(19) United States
(12) Patent Application Publication
(10) Pub. No.: US 2005/0137230 A1
(43) Pub. Date: Jun. 23, 2005

(54) Carboxamide derivatives

(76) Inventors: Dieter Dorsch, Ober-Ramstadt (DE); Werner Mederski, Zwingenberg (DE); Christos Tsaklakidis, Weinheim (DE); Bertram Cezanne, Morfelden-Walldorf (DE); Johannes Gleitz, Darmstadt (DE); Christopher Barnes, Bad Soden (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/059,655

(22) Filed: Feb. 17, 2005

Related U.S. Application Data
(62) Division of application No. 10/450,651, filed on Jun. 16, 2003.

(30) Foreign Application Priority Data
Dec. 16, 2000 (DE) 100 63 008.1

(51) Int. Cl. A61K 31/445; A61K 31/44; A61K 31/17

(52) U.S. Cl. 514/317; 514/357; 514/597

(57) ABSTRACT

Novel compounds of the formula I 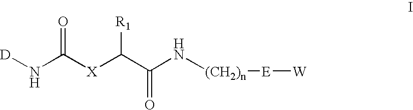 in which
R¹, D, E, W, X and n are as defined in claim 1, are inhibitors of coagulation factor Xa and can be employed for the prophylaxis and/or therapy of thromboembolic diseases and for the treatment of tumours.

This application is a divisional of U.S. application Ser. No. 10/450,651, filed 16 Jun. 2003, which was the National Stage of International App. No.

PCT/EP01/13545 filed 21 Nov. 2001.

The invention relates to compounds of the formula I 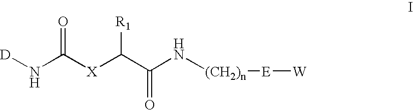
in which
D is phenyl or pyridyl, each of which is unsubstituted or monosubstituted or polysubstituted by Hal, A, OR², N(R²)₂, NO₂, CN, COOR² or CON(R²)₂,
R¹ is H, Ar, Het, cycloalkyl or A, which may be substituted by OR², SR², N(R²)₂, Ar, Het, cycloalkyl, CN, COOR² or CON(R²)₂,
R² is H or A,
E is phenylene, which may be monosubstituted or polysubstituted by Hal, A, OR², N(R²)₂, NO₂, CN, COOR² or CON(R²)₂, or is piperidine-1,4-diyl,
W is Ar, Het or N(R²)₂ and, if E=piperidine-1,4-diyl, is alternatively R² or cycloalkyl,
X is NH or O,
A is unbranched or branched alkyl having 1-10 carbon atoms, in which one or two CH₂ groups may be replaced by O or S atoms and/or by —CH═CH— groups and/or in addition 1-7H atoms may be replaced by F,
Ar is phenyl which is unsubstituted or monosubstituted, disubstituted or trisubstituted by Hal, A, OR², N(R²)₂, NO₂, CN, COOR², CON(R²)₂, NR²COA, NR²SO₂A, COR², SO₂NR², SO₃H or S(O)ₘA,
Het is a monocyclic or bicyclic, saturated, unsaturated or aromatic heterocyclic radical having from 1 to 4 N, O and/or S atoms, which may be unsubstituted or monosubstituted, disubstituted or trisubstituted by Hal, A, OR², N(R²)₂, NO₂, CN, COOR², CON(R²)₂, NR²COA, NR²SO₂A, COR², SO₂NR², SO₃H, S(O)ₘA and/or carbonyl oxygen,
Hal is F, Cl, Br or I,
n is 0 or 1,
m is 0, 1 or 2,
   and their pharmaceutically usable derivatives, solvates and stereoisomers, including mixtures thereof in all ratios.

The invention also relates to the optically active forms, the racemates, the diastereomers and the hydrates and solvates, for example alcoholates, of these compounds.

The invention had the object of finding novel compounds having valuable properties, in particular those which can be used for the preparation of medicaments.

It has been found that the compounds of the formula I and their salts have very valuable pharmacological properties and are well tolerated. In particular, they exhibit factor Xa-inhibiting properties and can therefore be employed for combating and preventing thromboembolic illnesses, such as thrombosis, myocardial infarction, arteriosclerosis, inflammation, apoplexia, angina pectoris, restenosis after angioplasty and claudicatio intermittens.

The compounds of the formula I according to the invention are furthermore inhibitors of the coagulation factors factor VIIa, factor IXa and thrombin in the blood coagulation cascade.

Other aromatic amides are described in WO 99/00121 and in WO 00/39118. Aromatic amidine derivatives having an antithrombotic action are disclosed, for example, in EP 0 540 051 B1. cyclic guanidines for the treatment of thromboembolic illnesses are described, for example, in WO 97/08165.

Aromatic heterocyclic compounds having factor Xa-inhibitory activity are disclosed, for example, in WO 96/10022. Substituted N-[(aminoimino-methyl)phenylalkyl]azaheterocyclylamides as factor Xa inhibitors are described in WO 96/40679.

The antithrombotic and anticoagulant effect of the compounds according to the invention is attributed to the inhibitory action against activated coagulation protease, known by the name factor Xa, or to the inhibition of other activated serine proteases, such as factor VIIa, factor IXa or thrombin.

Factor Xa is one of the proteases involved in the complex process of blood coagulation. Factor Xa catalyses the conversion of prothrombin into thrombin. Thrombin cleaves fibrinogen into fibrin monomers, which, after crosslinking, make an elementary contribution to thrombus formation. Activation of thrombin may result in the occurrence of thromboembolic illnesses. However, inhibition of thrombin may inhibit the fibrin formation involved in thrombus formation. The inhibition of thrombin can be measured, for example, by the method of G. F. Cousins et al. in Circulation 1996, 94, 1705-1712.

Inhibition of factor Xa can thus prevent the formation of thrombin.

The compounds of the formula I according to the invention and their salts engage in the blood coagulation process by inhibiting factor Xa and thus inhibit the formation of thrombuses.

The inhibition of factor Xa by the compounds according to the invention and the measurement of the anticoagulant and antithrombotic activity can be determined by conventional in-vitro or in-vivo methods. A suitable method is described, for example, by J. Hauptmann et al. in Thrombosis and Haemostasis 1990, 63, 220-223.

The inhibition of factor Xa can be measured, for example, by the method of T. Hara et al. in Thromb. Haemostas. 1994, 71, 314-319.

Coagulation factor VIIa initiates the extrinsic part of the coagulation cascade after binding to tissue factor and contributes to the activation of factor X to give factor Xa. Inhibition of factor VIIa thus prevents the formation of factor Xa and thus subsequent thrombin formation.

The inhibition of factor VIIa by the compounds according to the invention and the measurement of the anticoagulant and antithrombotic activity can be determined by conventional in-vitro or in-vivo methods. A conventional method for the measurement of the inhibition of factor VIIa is described, for example, by H. F. Ronning et al. in Thrombosis Research 1996, 84, 73-81.

Coagulation factor IXa is generated in the intrinsic coagulation cascade and is likewise involved in the activation of factor X to give factor Xa. Inhibition of factor IXa can therefore prevent the formation of factor Xa in a different way.

The inhibition of factor IXa by the compounds according to the invention and the measurement of the anticoagulant and antithrombotic activity can be determined by conventional in-vitro or in-vivo methods. A suitable method is described, for example, by J. Chang et al. in Journal of Biological Chemistry 1998, 273, 12089-12094.

The compounds according to the invention may furthermore be used for the treatment of tumours, tumour illnesses and/or tumour metastases.

A correlation between tissue factor TF/factor VIIa and the development of various types of cancer has been indicated by T. Taniguchi and N. R. Lemoine in Biomed. Health Res. (2000), 41 (Molecular Pathogenesis of Pancreatic Cancer), 57-59.

The publications listed below describe an antitumoural action of TF-VII and factor Xa inhibitors for various types of tumour:
K. M. Donnelly et al. in Thromb. Haemost. 1998; 79: 1041-1047;
E. G. Fischer et al. in J. Clin. Invest. 104: 1213-1221 (1999);
B. M. Mueller et al. in J. Clin. Invest. 101: 1372-1378 (1998);
M. E. Bromberg et al. in Thromb. Haemost. 1999; 82: 88-92

The compounds of the formula I can be employed as medicament active ingredients in human and veterinary medicine, in particular for the treatment and prevention of thromboembolic illnesses, such as thrombosis, myocardial infarction, arteriosclerosis, inflammation, apoplexia, angina pectoris, restenosis after angioplasty, claudicatio intermittens, venous thrombosis, pulmonary embolism, arterial thrombosis, myocardial ischaemia, unstable angina and strokes based on thrombosis.

The compounds according to the invention are also employed for the treatment or prophylaxis of atherosclerotic diseases, such as coronary arterial disease, cerebral arterial disease or peripheral arterial disease.

The compounds are also employed in combination with other thrombolytic agents in the case of myocardial infarction, furthermore for prophylaxis for reocclusion after thrombolysis, percutaneous transluminal angioplasty (PTCA) and coronary bypass operations.

The compounds according to the invention are furthermore used for the prevention of rethrombosis in microsurgery, furthermore as anticoagulants in connection with artificial organs or in haemodialysis.

The compounds are furthermore used in the cleaning of catheters and medical aids in vivo in patients, or as anticoagulants for the preservation of blood, plasma and other blood products in vitro. The compounds according to the invention are furthermore used for illnesses in which blood coagulation makes a crucial contribution to the course of the illness or represents a source of secondary pathology, such as, for example, in cancer, including metastasis, inflammatory disorders, including arthritis, and diabetes.

In the treatment of the illnesses described, the compounds according to the invention are also employed in combination with other thrombolytically active compounds, such as, for example, with “tissue plasminogen activator” t-PA, modified t-PA, streptokinase or urokinase. The compounds according to the invention are given either at the same time as or before or after the other substances mentioned.

Particular preference is given to simultaneous administration with aspirin in order to prevent recurrence of the clot formation.

The compounds according to the invention are also used in combination with blood platelet glycoprotein receptor (IIb/IIIa) antagonists, which inhibit blood platelet aggregation.

The invention relates to the compounds of the formula I and their salts and to a process for the preparation of the compounds of the formula I according to claim 1 and their salts, characterised in that
a) a compound of the formula II 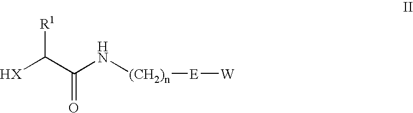
   in which
R¹, E, W, X and n are as defined in claim 1,
   is reacted with a compound of the formula III
   D-N═C═O III
   in which
D is as defined in claim 1,
   or
b) a compound of the formula IV
   H₂N—(CH₂)—E—W IV,
in which E, W and n are as defined in claim 1,
is reacted with a compound of the formula V 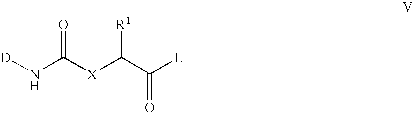
   in which
L is Cl, Br, I or a free or reactively functionally modified OH group, and R¹, X and D are as defined in claim 1,
   or
d) compounds of the formula I are liberated from one of their functional derivatives by treatment with a solvolysing or hydrogenolysing agent,
   or
c) a base or acid of the formula I is converted into one of its salts.

The invention also relates to the optically active forms (stereoisomers), the enantiomers, the racemates, the diastereomers and the hydrates and solvates of these compounds. The term solvates of the compounds is taken to mean adductions of inert solvent molecules onto the compounds which form owing to their mutual attractive force. Solvates are, for example, monohydrates or dihydrates or alcoholates.

The term pharmaceutically usable derivatives is taken to mean, for example, the salts of the compounds according to the invention and also so-called prodrug compounds.

The term prodrug derivatives is taken to mean, for example, compounds of the formula I which have been modified with, for example, alkyl or acyl groups, sugars or oligopeptides and which are rapidly cleaved in the organism to give the effective compounds according to the invention. These also include biodegradable polymer derivatives of the compounds according to the invention, as described, for example, in Int. J. Pharm. 115, 61-67 (1995).

The invention also relates to mixtures of the compounds of the formula I according to the invention, for example mixtures of two diastereomers, for example in the ratio 1:1, 1:2, 1:3, 1:4, 1:5, 1:10, 1:100 or 1:1000. These are particularly preferably mixtures of stereoisomeric compounds.

For all radicals which occur more than once, their meanings are independent of one another.

Above and below, the radicals or parameters R¹, D, E, W and n are as defined under the formula I, unless expressly stated otherwise.

A is alkyl, is unbranched (linear) or branched, and has 1, 2, 3, 4, 5, 6, 7, 8, 9 or 10 carbon atoms. A is preferably methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, furthermore preferably, for example, trifluoromethyl. A is very particularly preferably alkyl having 1-6 carbon atoms, preferably methyl, ethyl, propyl, isopropyl, butyl, isobutyl, sec-butyl, tert-butyl, pentyl, hexyl or trifluoromethyl.

Cycloalkyl is preferably, for example, cyclopropyl, cyclobutyl, cyclopentyl, cyclohexyl or cycloheptyl.

Hal is preferably F, Cl or Br, but also 1.

Ar is, for example, phenyl, o-, m- or p-tolyl, o-, m- or p-ethylphenyl, o-, m- or p-propylphenyl, o-, m- or p-isopropylphenyl, o-, m- or p-tert-butylphenyl, o-, m- or p-hydroxyphenyl, o-, m- or p-nitrophenyl, o-, m- or p-aminophenyl, o-, m- or p-(N-methylamino)phenyl, o-, m- or p-(N-methylaminocarbonyl)phenyl, o-, m- or p-acetamidophenyl, o-, m- or p-methoxyphenyl, o-, m- or p-ethoxy-phenyl, o-, m- or p-ethoxycarbonylphenyl, o-, m- or p-(N,N-dimethylamino)-phenyl, o-, m- or p-(N,N-dimethylaminocarbonyl)phenyl, o-, m- or p-(N-ethyl-amino)-phenyl, o-, m- or p-(N,N-diethylamino)-phenyl, o-, m- or p-fluoro-phenyl, o-, m- or p-bromophenyl, o-, m- or p-chlorophenyl, o-, m- or p-(methylsulfonamido)phenyl, o-, m- or p-(methylsulfonyl)-phenyl, further preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromo-phenyl, 2,4- or 2,5-dinitrophenyl, 2,5- or 3,4-dimethoxyphenyl, 3-nitro-4-chlorophenyl, 3-amino-4-chloro-, 2-amino-3-chloro-, 2-amino-4-chloro-, 2-amino-5-chloro- or 2-amino-6-chlorophenyl, 2-nitro-4-N,N-dimethylamino- or 3-nitro-4-N,N-dimethylaminophenyl, 2,3-diaminophenyl, 2,3,4-, 2,3,5-, 2,3,6-, 2,4,6- or 3,4,5-trichloroophenyl, 2,4,6-trimethoxyphenyl, 2-hydroxy-3,5-dichlorophenyl, p-iodophenyl, 3,6-dichloro-4-aminophenyl, 4-fluoro-3-chlorophenyl, 2-fluoro-4-bromophenyl, 2,5-difluoro-4-bromophenyl, 3-bromo-6-methoxyphenyl, 3-chloro-6-methoxyphenyl, 3-chloro-4-acetamidophenyl, 3-fluoro-4-methoxyphenyl, 3-amino-6-methylphenyl, 3-chloro-4-acetamidophenyl or 2,5-dimethyl-4-chlorophenyl.

Ar is preferably, for example, phenyl which is unsubstituted or monosubstituted, disubstituted or trisubstituted by Hal, A, OR², SO₂A, COOR² or CN phenyl.

Ar is particularly preferably, for example, phenyl which is unsubstituted or monosubstituted or disubstituted by Hal, A, OA, SO₂A, SO₂NH₂, COOR² or CN, such as, for example, phenyl, 2-methylsulfonylphenyl, 2-aminosulfonyl-phenyl, 2-, 3- or 4-chlorophenyl, 4-methylphenyl, 4-bromophenyl, 3-fluoro-4-methoxyphenyl, 4-trifluoromethoxyphenyl, 4-ethoxyphenyl, 2-methoxyphenyl, 3-cyanophenyl or 4-ethoxycarbonylphenyl.

Ar is very particularly preferably unsubstituted phenyl, 4-chlorophenyl or 2-methylsulfonylphenyl.

Het is, for example, 2- or 3-furyl, 2- or 3-thienyl, 1-, 2- or 3-pyrrolyl, 1-, 2-, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or 5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-quinolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, furthermore preferably 1,3-benzodioxol-5-yl, 1,4-benzodioxan-6-yl, 2,1,3-benzothiadiazol-4- or -5-yl or 2,1,3-benzoxadiazol-5-yl.

The heterocyclic radicals may also be partially or fully hydrogenated. Het can thus, for example, also be 2,3-dihydro-2-, -3-, -4- or -5-furyl, 2,5-dihydro-2-, -3-, -4- or 5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2-or-4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-,-2-,-3-, -4-, -5-, -6-, -7- or -8-isoquinolyl, 2-, 3-, 5-, 6-, 7- or 8-3,4-dihydro-2H-benzo-1,4-oxazinyl, furthermore preferably 2,3-methylenedioxyphenyl, 3,4-methylenedioxyphenyl, 2,3-ethylenedioxy-phenyl, 3,4-ethylenedioxyphenyl, 3,4-(difluoromethylenedioxy)phenyl, 2,3-dihydrobenzofuran-5- or 6-yl, 2,3-(2-oxo-methylenedioxy)phenyl or alternatively 3,4-dihydro-2H-1,5-benzodioxepin-6- or -7-yl, furthermore preferably 2,3-dihydrobenzofuranyl or 2,3-dihydro-2-oxofuranyl.

Het is preferably a monocyclic or bicyclic, saturated, unsaturated or aromatic heterocyclic radical having from 1 to 2 N, O and/or S atoms, which may be unsubstituted or monosubstituted by carbonyl oxygen.

Het is preferably, for example, furyl, thienyl, thiazolyl, imidazolyl, 2,1,3-benzothiadiazolyl, oxazolyl, pyridyl, indolyl, piperidinyl, morpholinyl, tetrahydropyranyl, piperazinyl, pyrazinyl, piperidinyl or pyrrolidinyl, optionally substituted by carbonyl oxygen, such as, for example, 3-oxomorpholin-4-yl, 2-oxopiperidin-1-yl or 2-oxopyrrolidin-1-yl.

Het is very particularly preferably thienyl, imidazolyl, pyridyl, indolyl, piperidinyl, piperazinyl, 2-oxopiperazinyl, morpholinyl, tetrahydropyran-4-yl, 3-oxomorpholin-4-yl, 2-oxo-2H-pyrazin-1-yl, 2-oxo-pyrrolidin-1-yl or 2-oxopiperidin-1-yl.

D is in particular, for example, phenyl which is unsubstituted or monosubstituted or disubstituted by Hal, A, hydroxyl, methoxy, ethoxy, hydroxycarbonyl, methoxycarbonyl or ethoxycarbonyl, or pyridyl which is unsubstituted or monosubstituted by Hal.

D is very particularly preferably 4-chlorophenyl or 3-chloro-2-pyridyl.

R¹ is preferably, for example, H, phenyl or alkyl having 1-6 carbon atoms, which may be substituted by thiophene, imidazole, indole, SR², cycloalkyl or phenyl.

R¹ is in particular, for example, H, methyl, ethyl, propyl, butyl, tert-butyl, pentyl, cyclopropylmethyl, thiophen-2-ylmethyl, imidazol-4-ylmethyl, methylsulfanylethyl, phenyl, benzyl, pyridin-3-ylmethyl, indol-3-ylmethyl, aminopropyl or 3-cyanobenzyl, furthermore pyridin-2-yl, 2- or 4-fluorophenyl or 4-hydroxyphenyl.

R² is preferably, for example, H or alkyl having 1, 2, 3, 4, 5 or 6 carbon atoms.

n is preferably 0 or 1.

m is preferably 2.

E is preferably, for example, 1,4-phenylene or 1,4-piperidinyl.

W is preferably, for example, 2-methylsulfonylphenyl, 4-pyridinyl, tetrahydropyran-4-yl, 2-oxopiperidin-1-yl, 3-oxomorpholin-4-yl, dimethylamino, diethylamino, piperazinyl, morpholin-4-yl, 2-oxopyrrolidin-1-yl, piperidin-1- or-4-yl or phenyl.

If W is 1,3-piperidinyl, W is preferably alternativley, for example, isopropyl, cyclopentyl or cyclohexyl.

The compounds of the formula I may have one or more chiral centres and therefore occur in various stereoisomeric forms. The formula I covers all these forms.

Accordingly, the invention relates in particular to the compounds of the formula I in which at least one of the said radicals has one of the preferred meanings indicated above. Some preferred groups of compounds may be expressed by the following sub-formulae Ia to Im, which conform to the formula I and in which the radicals not designated in greater detail are as defined under the formula I, but in which

| | | |
|---|---|---|
| in la | D | is phenyl which is unsubstituted or monosubstituted or |
| | | disubstituted by Hal, A, OR² or COOR², or pyridyl which is |
| | | unsubstituted or monosubstituted by Hal; |
| in lb | Het | is a monocyclic or bicyclic, saturated, unsaturated or |
| | | aromatic heterocyclic radical having from 1 to 2 N, O and/or |
| | | S atoms, which may be unsubstituted or monosubstituted by |
| | | carbonyl oxygen; |
| in lc | Ar | is phenyl which is unsubstituted or monosubstituted, |
| | | disubstituted or trisubstituted by Hal, A, OR², SO₂A, |
| | | SO₂NH₂, COOR² or CN; |
| in ld | D | is phenyl which is unsubstituted or monosubstituted or |
| | | disubstituted by Hal, A, hydroxyl, methoxy, ethoxy, hydroxycarbonyl, |
| | | methoxycarbonyl or ethoxycarbonyl, or pyridyl |
| | | which is unsubstituted or monosubstituted by Hal; |
| in le | R¹ | is H, phenyl or alkyl having 1-6 carbon atoms, which may be |
| | | substituted by thiophene, imidazole, indole, SR², cycloalkyl |
| | | or phenyl; |
| in lf | E | is 1,4-phenylene or 1,4-piperidinyl; |
| in lg | Ar | is phenyl which is unsubstituted or monosubstituted, |
| | | disubstituted or trisubstituted by Hal, A, OR², SO₂A, |
| | | SO₂NH₂, COOR² or CN, |
| | Het | is a monocyclic or bicyclic, saturated, unsaturated or |
| | | aromatic heterocyclic radical having from 1 to 2 N, O and/or |
| | | S atoms, which may be unsubstituted or monosubstituted by |
| | | carbonyl oxygen, |
| | W | is Ar, Het or N(R²)₂ |
| | | and, if E = piperidine-1,4-diyl, is alternatively R²; |
| in lh | Ar | is phenyl which is unsubstituted or monosubstituted or |
| | | disubstituted by Hal, A, OA, SO₂A, COOR², SO₂NH₂ or CN, |
| | Het | is thienyl, imidazolyl, pyridyl, indolyl, piperidinyl, piperazinyl, |
| | | 2-oxopiperazinyl, pyrazinyl, 2-oxo-2H-pyrazin-1-yl, |
| | | morpholinyl, tetrahydropyran-4-yl, 3-oxomorpholin-4-yl, 2- |
| | | oxopyrrolidin-1-yl or 2-oxopiperidin-1-yl, |
| | W | is Ar, Het or N(R²)₂ |
| | | and, if E = piperidine-1,4-diyl, is alternatively R²; |
| in li | D | is phenyl which is unsubstituted or monosubstituted or |
| | | disubstituted by Hal, A, OR² or COOR², or pyridyl which is |
| | | unsubstituted or monosubstituted by Hal, |
| | R¹ | is H, phenyl or alkyl having 1-6 carbon atoms, which may be |
| | | substituted by thiophene, imidazole, indole, SR², cycloalkyl |
| | | or phenyl, |
| | R² | is H or A, |
| | E | is 1,4-phenylene or 1,4-piperidinyl, |
| | W | is Ar, Het or N(R₂)₂ |
| | | and, if E = piperidine-1,4-diyl, is alternatively R², |
| | A | is alkyl having 1, 2, 3, 4, 5 or 6 carbon atoms or CF₃, |
| | Ar | is phenyl which is unsubstituted or monosubstituted or |
| | | disubstituted by Hal, A, OA, SO₂A, COOR², SO₂NH₂ or CN, |
| | Het | is thienyl, imidazolyl, pyridyl, indolyl, piperidinyl, piperazinyl, |
| | | 2-oxopiperazinyl, pyrazinyl, 2-oxo-2H-pyrazin-1-yl, |
| | | morpholinyl, tetrahydropyran-4-yl, 3-oxomorpholin-4-yl, 2- |
| | | oxopyrrolidin-1-yl or 2-oxopiperidin-1-yl, |
| | Hal | is F, Cl or Br, |
| | n | is 0 or 1, |
| | m | is 1 or 2; |
| in lj | D | is phenyl which is unsubstituted or monosubstituted by Hal, |
| | | or pyridyl which is unsubstituted or monosubstituted by Hal, |
| | R¹ | is H, phenyl or alkyl having 1-6 carbon atoms, which may be |
| | | substituted by thiophene, imidazole, indole, SR², cycloalkyl |
| | | or phenyl, |
| | R² | is H or A, |
| | E | is 1,4-phenylene, |
| | W | is 2-methylsulfonylphenyl, |
| | X | is NH or O, |
| | A | is alkyl having 1, 2, 3, 4, 5 or 6 carbon atoms or CF₃, |
| | n | is 0, |
| | | and their pharmaceutically tolerated salts and solvates; |
| in lk | D | is phenyl which is unsubstituted or monosubstituted by Hal, |
| | | or pyridyl which is unsubstituted or monosubstituted by Hal, |
| | R¹ | is H, phenyl or alkyl having 1-6 carbon atoms, which may be |
| | | substituted by thiophene, imidazole, indole, SR², cycloalkyl |
| | | or phenyl, |
| | R² | is H or A, |
| | E | is 1,4-piperidinyl, |
| | W | is Het, |
| | Het | is thienyl, imidazolyl, pyridyl, indolyl, piperidinyl, morpholinyl, |
| | | piperazinyl, 2-oxopiperazinyl, pyrazinyl, 2-oxo-2H-pyrazin-1- |
| | | yl, tetrahydropyran-4-yl, 3-oxo-morpholin-4-yl or 2- |
| | | oxopiperidin-1-yl, |
| | X | is NH or O, |
| | A | is alkyl having 1, 2, 3, 4, 5 or 6 carbon atoms or CF₃, |
| | n | is 0 or 1; |
| in ll | R¹ | is H, phenyl or alkyl having 1-6 carbon atoms, which may be |
| | | substituted by thiophene, imidazole, indole, SR², cycloalkyl |
| | | or phenyl, |
| | | or phenyl or pyridyl, each of which is monosubstituted by Hal |
| | | or OH; |
| in lm | D | is phenyl which is unsubstituted or monosubstituted by Hal, |
| | | or pyridyl which is unsubstituted or monosubstituted by Hal, |
| | R¹ | is H, phenyl or alkyl having 1-6 carbon atoms, which may be |
| | | substituted by thiophene, imidazole, indole, SR², cycloalkyl |
| | | or phenyl, |
| | R² | is H or A, |
| | E | is 1,4-piperidinyl, |
| | W | is Het, R² or cycloalkyl, |
| | Het | is thienyl, imidazolyl, pyridyl, indolyl, piperidinyl, piperazinyl, |
| | | 2-oxopiperazinyl, pyrazinyl, 2-oxo-2H-pyrazin-1-yl, |
| | | morpholinyl, tetrahydropyran-4-yl, 3-oxomorpholin-4-yl or 2- |
| | | oxo-piperidin-1-yl, |
| | X | is NH or O, |
| | A | is alkyl having 1, 2, 3, 4, 5 or 6 carbon atoms or CF₃, |
| | n | is 0 or 1, |
| | | and their pharmaceutically usable derivatives, solvates and stereoisomers, |
| | | including mixtures thereof in all ratios. |

The compounds of the formula I and also the starting materials for the preparation are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants which are known per se, but are not mentioned here in greater detail.

If desired, the starting materials can also be formed in situ so that they are not isolated from the reaction mixture, but instead are immediately converted further into the compounds of the formula I.

Compounds of the formula I can preferably be obtained by reacting compounds of the formula II with compounds of the formula III.

The reaction is generally carried out in an inert solvent, in the presence of an acid-binding agent, preferably an alkali or alkaline earth metal hydroxide, carbonate or bicarbonate or another salt of a weak acid of the alkali or alkaline earth metals, preferably of potassium, sodium, calcium or caesium. The addition of an organic base, such as triethylamine, dimethylaniline, pyridine or quinoline, may also be favourable. Depending on the conditions used, the reaction time is between a few minutes and 14 days, and the reaction temperature is between about 0° and 1500, normally between 20° and 130°.

Examples of suitable inert solvents are water, hydrocarbons, such as hexane, petroleum ether, benzene, toluene or xylene; chlorinated hydrocarbons, such as trichloroethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

The starting compounds of the formulae II and III are generally known. If they are novel, they can, however, be prepared by methods known per se.

Compounds of the formula I can also be obtained by reacting compounds of the formula IV with compounds of the formula V. In the compounds of the formula V, L is preferably Cl, Br, I or a reactively modified OH group, such as, for example, an activated ester, an imidazolide or alkylsulfonyloxy having 1-6 carbon atoms (preferably methylsulfonyloxy or trifluoromethyl-sulfonyloxy) or arylsulfonyloxy having 6-10 carbon atoms (preferably phenyl- or p-tolylsulfonyloxy).

The reaction is generally carried out in an inert solvent, in the presence of an acid-binding agent, preferably an alkali or alkaline earth metal hydroxide, carbonate or bicarbonate or another salt of a weak acid of the alkali or alkaline earth metals, preferably of potassium, sodium, calcium or caesium. The addition of an organic base, such as triethylamine, dimethylaniline, pyridine or quinoline or an excess of the amine component of the formula IV, may also be favourable. Depending on the conditions used, the reaction time is between a few minutes and 14 days, and the reaction temperature is between about 0° and 150°, normally between 200 and 130°.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

Compounds of the formula I can also be obtained by liberating compounds of the formula I from one of their functional derivatives by treatment with a solvolysing or hydrogenolysing agent.

Preferred starting materials for the solvolysis or hydrogenolysis are those which conform to the formula I, but contain corresponding protected amino and/or hydroxyl groups instead of one or more free amino and/or hydroxyl groups, preferably those which carry an amino-protecting group instead of an H atom bonded to an N atom, in particular those which carry an R′—N group, in which R′ is an amino-protecting group, instead of an HN group, and/or those which carry an hydroxyl-protecting group instead of the H atom of an hydroxyl group, for example those which conform to the formula I, but carry a —COOR″ group, in which R″ is an hydroxyl-protecting group, instead of a —COOH group.

It is also possible for a plurality of—identical or different—protected amino and/or hydroxyl groups to be present in the molecule of the starting material. If the protecting groups present are different from one another, they can in many cases be cleaved off selectively.

The term “amino-protecting group” is known in general terms and relates to groups which are suitable for protecting (blocking) an amino group against chemical reactions, but which are easy to remove after the desired chemical reaction has been carried out elsewhere in the molecule. Typical of such groups are, in particular, unsubstituted or substituted acyl, aryl, aralkoxymethyl or aralkyl groups. Since the amino-protecting groups are removed after the desired reaction (or reaction sequence), their type and size is furthermore not crucial; however, preference is given to those having 1-20, in particular 1-8, carbon atoms. The term “acyl group” is to be understood in the broadest sense in connection with the present process. It includes acyl groups derived from aliphatic, araliphatic, aromatic or heterocyclic carboxylic acids or sulfonic acids, and, in particular, alkoxycarbonyl, aryloxycarbonyl and especially aralkoxycarbonyl groups. Examples of such acyl groups are alkanoyl, such as acetyl, propionyl and butyryl; aralkanoyl, such as phenylacetyl; aroyl, such as benzoyl and tolyl; aryloxyalkanoyl, such as POA; alkoxycarbonyl, such as methoxycarbonyl, ethoxycarbonyl, 2,2,2-trichloroethoxycarbonyl, BOC (tert-butoxycarbonyl) and 2-iodoethoxycarbonyl; aralkoxycarbonyl, such as CBZ (“carbobenzoxy”), 4-methoxybenzyloxycarbonyl and FMOC; and arylsulfonyl, such as Mtr. Preferred amino-protecting groups are BOC and Mtr, furthermore CBZ, Fmoc, benzyl and acetyl.

The compounds of the formula I are liberated from their functional derivatives—depending on the protecting group used—for example using strong acids, advantageously using TFA or perchloric acid, but also using other strong inorganic acids, such as hydrochloric acid or sulfuric acid, strong organic carboxylic acids, such as trichloroacetic acid, or sulfonic acids, such as benzene- or p-toluenesulfonic acid. The presence of an additional inert solvent is possible, but is not always necessary. Suitable inert solvents are preferably organic, for example carboxylic acids, such as acetic acid, ethers, such as tetrahydrofuran or dioxane, amides, such as DMF, halogenated hydrocarbons, such as dichloromethane, furthermore also alcohols, such as methanol, ethanol or isopropanol, and water. Mixtures of the above-mentioned solvents are furthermore suitable. TFA is preferably used in excess without addition of a further solvent, and perchloric acid is preferably used in the form of a mixture of acetic acid and 70% perchloric acid in the ratio 9:1. The reaction temperatures for the cleavage are advantageously between about 0 and about 50°, preferably between 15 and 30° (room temperature).

The BOC, OBut and Mtr groups can, for example, preferably be cleaved off using TFA in dichloromethane or using approximately 3 to 5N HCl in dioxane at 15-30°, and the FMOC group can be cleaved off using an approximately 5 to 50% solution of dimethylamine, diethylamine or piperidine in DMF at 15-30°.

Protecting groups which can be removed hydrogenolytically (for example CBZ, benzyl or the liberation of the amidino group from its oxadiazole derivative) can be cleaved off, for example, by treatment with hydrogen in the presence of a catalyst (for example a noble-metal catalyst, such as palladium, advantageously on a support, such as carbon). Suitable solvents here are those indicated above, in particular, for example, alcohols, such as methanol or ethanol, or amides, such as DMF. The hydrogenolysis is generally carried out at temperatures between about 0 and 100° and pressures between about 1 and 200 bar, preferably at 20-30° and 1-10 bar. Hydrogenolysis of the CBZ group succeeds well, for example, on 5 to 10% Pd/C in methanol or using ammonium formate (instead of hydrogen) on Pd/C in methanol/DMF at 20-30°.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, trifluoromethylbenzene, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, N-methylpyrrolidone (NMP) or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

The biphenyl-SO₂NH₂ group is preferably employed in the form of its tert-butyl derivative. The tert-butyl group is cleaved off, for example, using TFA with or without addition of an inert solvent, preferably with addition of a small amount of anisole (1-10% by volume).

It is furthermore possible to convert a compound of the formula I into another compound of the formula I by converting one or more R¹, D, E and/or W radical(s) into one or more R¹, D, E and/or W radical(s), for example by acylating an amino group or reducing nitro groups to amino groups (for example by hydrogenation on Raney nickel or Pd/carbon in an inert solvent, such as methanol or ethanol).

Esters can be saponified, for example, using acetic acid or using NaOH or KOH in water, water/THF or water/dioxane, at temperatures between 0 and 100°.

Free amino groups can furthermore be acylated in a conventional manner using an acid chloride or anhydride or alkylated using an unsubstituted or substituted alkyl halide, advantageously in an inert solvent, such as dichloro-methane or THF and/or in the presence of a base, such as triethylamine or pyridine, at temperatures between −60 and +30°.

If W is 1,4-piperidinyl, the alkylation of the piperidine nitrogen can be carried out by conventional methods of reductive amination.

A base of the formula I can be converted into the associated acid-addition salt using an acid, for example by reaction of equivalent amounts of the base and the acid in an inert solvent, such as ethanol, followed by evaporation. Suitable acids for this reaction are, in particular, those which give physiologically acceptable salts. Thus, it is possible to use inorganic acids, for example sulfuric acid, nitric acid, hydrohalic acids, such as hydrochloric acid or hydrobromic acid, phosphoric acids, such as ortho-phosphoric acid, or sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono- and -disulfonic acids, and laurylsulfuric acid. Salts with physiologically unacceptable acids, for example picrates, can be used for the isolation and/or purification of the compounds of the formula I.

On the other hand, compounds of the formula I can be converted into the corresponding metal salts, in particular alkali metal or alkaline earth metal salts, or into the corresponding ammonium salts using bases (for example sodium hydroxide, potassium hydroxide, sodium carbonate or potassium carbonate). It is also possible to use physiologically acceptable organic bases, such as, for example, ethanolamine.

Compounds of the formula I according to the invention may be chiral owing to their molecular structure and may accordingly occur in various enantiomeric forms. They can therefore exist in racemic or in optically active form.

Since the pharmaceutical activity of the racemates or stereoisomers of the compounds according to the invention may differ, it may be desirable to use the enantiomers. In these cases, the end product or even the intermediates can be separated into enantiomeric compounds by chemical or physical measures known to the person skilled in the art or even employed as such in the synthesis.

In the case of racemic amines, diastereomers are formed from the mixture by reaction with an optically active resolving agent. Examples of suitable resolving agents are optically active acids, such as the R and S forms of tartaric acid, diacetyltartaric acid, dibenzoyltartaric acid, mandelic acid, malic acid, lactic acid, suitable N-protected amino acids (for example N-benzoylproline) or N-benzenesulfonylproline), or the various optically active camphorsulfonic acids. Also advantage is chromatographic enantiomer resolution with the aid of an optically active resolving agent (for example dinitrobenzoylphenylglycine, cellulose triacetate or other derivatives of carbohydrates or chirally derivatised methacrylate polymers immobilised on silica gel). Examples of suitable eluents for this purpose are aqueous or alcoholic solvent mixtures, such as, for example, hexane/isopropanol/acetonitrile, for example in the ratio 82:15:3.

The invention furthermore relates to the use of compounds of the formula I and/or their physiologically acceptable salts for the preparation of pharmaceutical preparations, in particular by non-chemical methods. They can be converted here into a suitable dosage form together with at least one solid, liquid and/or semiliquid excipient or assistant and, if desired, in combination with one or more further active ingredients.

The invention furthermore relates to medicaments comprising at least one compound of the formula I and/or its pharmaceutically usable derivatives, solvates and stereoisomers, including mixtures thereof in all ratios, and optionally excipients and/or assistants.

These medicaments can be used in human or veterinary medicine. Suitable excipients are organic or inorganic substances which are suitable for enteral (for example oral), parenteral or topical administration and do not react with the novel compounds, for example water, vegetable oils, benzyl alcohols, alkylene glycols, polyethylene glycols, glycerol triacetate, gelatin, carbohydrates, such as lactose or starch, magnesium stearate, talc or vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders or also as nasal sprays. The novel compounds may also be lyophilised and the resultant lyophilisates used, for example, to prepare injection preparations. The preparations indicated may be sterilised and/or comprise assistants, such as lubricants, preservatives, stabilisers and/or wetting agents, emulsifying agents, salts for modifying the osmotic pressure, buffer substances, colorants and flavours and/or a plurality of further active ingredients, for example one or more vitamins.

The compounds of the formula I and their physiologically acceptable salts can be used for combating thromboembolic illnesses, such as thrombosis, myocardial infarction, arteriosclerosis, inflammation, apoplexia, angina pectoris, restenosis after angioplasty, claudicatio intermittens, tumours, tumour diseases and/or tumour metastases.

In general, the substances according to the invention are preferably administered in doses between about 1 and 500 mg, in particular between 5 and 100 mg, per dosage unit. The daily dose is preferably between about 0.02 and 10 mg/kg of body weight. However, the specific dose for each patient depends on a wide variety of factors, for example on the efficacy of the specific compound employed, on the age, body weight, general state of health, sex, on the diet, on the time and method of administration, on the excretion rate, medicament combination and severity of the particular illness to which the therapy applies. Oral administration is preferred.

The invention furthermore relates to medicaments comprising at least one compound of the formula I and/or its pharmaceutically usable derivatives, solvates and stereoisomers, including mixtures thereof in all ratios, and at least one further medicament active ingredient.

The invention also relates to a set (kit) consisting of separate packs of
(a) an effective amount of a compound of the formula I and/or its pharmaceutically usable derivatives, solvates and stereoisomers, including mixtures thereof in all ratios, and
(b) an effective amount of a further medicament active ingredient.

The set comprises suitable containers, such as boxes, individual bottles, bags or ampoules. The set may, for example, comprise separate ampoules each containing an effective amount of a compound of the formula I and/or its pharmaceutically usable derivatives, solvates and stereoisomers, including mixtures thereof in all ratios, and an effective amount of a further medicament active ingredient in dissolved or lyophilised form.

The invention furthermore relates to the use of compounds of the formula I and/or their pharmaceutically usable derivatives, solvates and stereoisomers, including mixtures thereof in all ratios, for the preparation of a medicament for the treatment of thrombosis, myocardial infarction, arteriosclerosis, inflammation, apoplexia, angina pectoris, restenosis after angioplasty, claudicatio intermittens, tumours, tumour diseases and/or tumour metastases, in combination with at least one further medicament active ingredient.

Above and below, all temperatures are given in ° C. In the following examples, ‘conventional work-up’ means that water is added if necessary, the pH is adjusted, if necessary, to between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallisation. Rf values on silica gel; eluent: ethyl acetate/methanol 9:1.

Mass spectrometry (MS): EI (electron ionisation) M⁺ESI (electrospray ionisation) (M+H)⁺FAB (fast atom bombardment) (M+H)⁺

### EXAMPLE 1

1.1 1.08 g of 4-methylmorpholine are added to a solution of 3.0 g of (R)-2-benzyloxycarbonylamino-3-phenylpropionic acid (Z-D-phenylalanine), 2.52 g of 2′-methylsulfonylbiphenyl-4-ylamine, 1.93 g of N-(3-dimethylaminopropyl)-N′-ethylcarbodiimide, hydrochloride (DAPECI) and 1.43 g of 1-hydroxybenzotriazole (HOBt) in 25 ml of DMF, and the mixture is stirred at room temperature for a further 40 hours. The reaction mixture is introduced into water, and the precipitate is filtered off, giving benzyl [(R)-1-(2′-methylsulfonylbiphenyl-4-ylcarbamoyl)-2-phenylethyl]carbamate (“AA”), ESI 529,

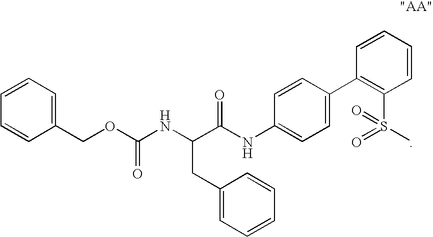

1.2 A solution of 4.39 g of “M” in 50 ml of methanol is hydrogenated using palladium on active carbon as catalyst. The catalyst is separated off, the solvent is removed, and the residue is chromatographed over a silica gel column (petroleum ether/ethyl acetate), giving (R)-2-amino-N-(2′-methylsulfonylbiphenyl-4-yl)-3-phenylpropionamide (“AB”), ESI 395,

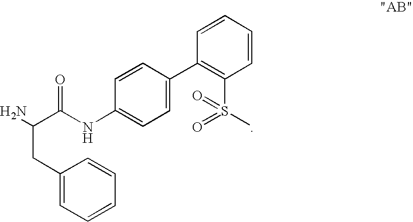

1.3 81 mg of 4-chlorophenyl isocyanate are added to a solution of 200 mg of “AB” in 5 ml of dichloromethane, and the mixture is stirred at room temperature for a further 4 hours. 200 mg of tris(2-aminoethyl)amine-polystyrene (polyamine resin) are subsequently added, the mixture is stirred at room temperature for a further 18 hours, and the resin is separated off. Removal of the solvent gives (R)-2-[3-(4-chlorophenyl)ureido]-N-(2′-methylsulfonylbiphenyl-4-yl)-3-phenylpropionamide, ESI 548, 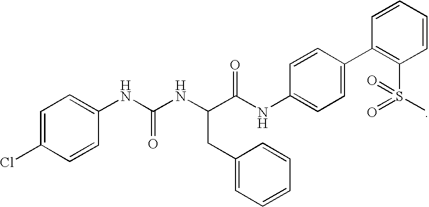
IC₅₀(Xa)=8.6×10⁻⁸ M;
IC₅₀ (VIIa)=6.5×10⁻⁸ M.

### EXAMPLE 2

2.1 A solution of 3.0 g of (R)-2-aminopropionic acid (D-alanine) and 5.63 g of sodium hydrogencarbonate in 50 ml of water is heated to 80°. 10.3 g of 4-chlorophenyl isocyanate are added, and the mixture is stirred at 80° for a further 1 hour. Conventional work-up gives (R)-2-[3-(4-chlorophenyl)-ureido]propionic acid (“BA”), ESI 243.

2.2 28 mg of 4-methylmorpholine are added to a solution of 68 mg of “BA”, 62 mg of 2′-methylsulfonylbiphenyl-4-ylamine (“BB”), 54 mg of DAPECI and 38 mg of HOBt in 1 ml of DMF, and the mixture is stirred at room temperature for 40 hours. The reaction mixture is introduced into water, and the precipitate is filtered off, giving (R)-2-[3-(4-chlorophenyl)ureido]-N-(2′-methylsulfonylbiphenyl-4-yl)-propionamide, ESI 472.

Analogous reaction of “BB” with
(S)-2-(3-pyridin-2-ylureido)pentanoic acid,
(R)-2-(3-phenylureido)pentanoic acid,
2-(3-phenylureido)-3-(thiophen-2-yl)propionic acid,
2-(3-phenylureido)-3-(3H-imidazol-4-yl )propionic acid,
2-(3-phenylureido)hexanoic acid,
2-(3-phenylureido)-4-(methylsulfanyl)butyric acid,
2-(3-phenylureido)-2-phenylacetic acid,
(S)-2-[3-(4-chlorophenyl)ureido]-3-phenylpropionic acid (R)-2-[3-(4-methylphenyl)ureido]-3-phenylpropionic acid (R)-2-(3-pyridin-4-ylureido)pentanoic acid,
(S)-2-(3-pyridin-4-ylureido)pentanoic acid,
(R)-2-(3-pyridin-2-ylureido)pentanoic acid,
(S)-2-(3-phenylureido)pentanoic acid,
(R)-2-(3-pyridin-3-ylureido)pentanoic acid,
(S)-2-(3-phenylureido)-3-(pyridin-3-yl)propionic acid,
(S)-2-(3-phenylureido)-3-(indol-3-yl)propionic acid,
2-(3-phenylureido)propionic acid,
2-(3-phenylureido)acetic acid,
(S)-2-[3-(3-chlorophenyl)ureido]-3-phenylpropionic acid,
(S)-2-[3-(4-trifluoromethylphenyl)ureido]-3-phenylpropionic acid,
(S)-2-[3-(2-chlorophenyl)ureido]-3-phenylpropionic acid,
(S)-2-[3-(4-ethoxyphenyl)ureido]-3-phenylpropionic acid,
(S)-2-[3-(4-methylphenyl)ureido]-3-phenylpropionic acid,
(S)-2-[3-(2-methoxyphenyl)ureido]-3-phenylpropionic acid,
(S)-2-[3-(4-ethoxycarbonylphenyl)ureido]-3-phenylpropionic acid,
(R)-2-[3-(3-chlorophenyl)ureido]-3-phenylpropionic acid,
(R)-2-[3-(4-trifluoromethylphenyl)ureido]-3-phenylpropionic acid,
(R)-2-[3-(2-chlorophenyl)ureido]-3-phenylpropionic acid,
(R)-2-[3-(4-ethoxyphenyl)ureido]-3-phenylpropionic acid,
(R)-2-[3-(2-methoxyphenyl)ureido]-3-phenylpropionic acid,
(R)-2-[3-(4-ethoxycarbonylphenyl)ureido]-3-phenylpropionic acid,
2-(3-phenylureido)-5-BOC-aminovaleric acid,
(S)-2-(3-phenylureido)-3-phenylpropionic acid,
(R)-2-(3-phenylureido)-3-phenylpropionic acid,
(R)-2-[3-(4-chlorophenyl)ureido]-3-cyclopropylpropionic acid,
2-[3-(4-chlorophenyl)ureido]-4-(methylsulfanyl)butyric acid,
(R)-2-[3-(4-chlorophenyl)ureido]propionic acid,
2-[3-(4-chlorophenyl)ureido]acetic acid,
(R)-2-[3-(5-chloro-pyridin-2-yl)ureido]-3-phenylpropionic acid,
(R)-2-[3-(4-bromophenyl)ureido]-3-phenylpropionic acid,
(R)-2-[3-(3-fluoro-4-methoxyphenyl)ureido]-3-phenylpropionic acid,
2-[3-(4-chlorophenyl)ureido]hexanoic acid,
(R)-2-[3-(4-chlorophenyl)ureido]-2-phenylacetic acid,
(S)-2-[3-(4-chlorophenyl)ureido]-4-methylpentanoic acid,
(R)-2-[3-(4-chlorophenyl)ureido]-4-methylpentanoic acid,
(S)-2-[3-(4-methoxyphenyl)ureido]-3-phenylpropionic acid,
(S)-2-[3-(4-bromophenyl)ureido]-3-phenylpropionic acid,
(S)-2-[3-(4-fluorophenyl)ureido]-3-phenylpropionic acid,
(S)-2-[3-(4-fluorophenyl)ureido]-3-phenylpropionic acid,
(S)-2-[3-(3-fluoro-4-methoxyphenyl)ureido]-3-phenylpropionic acid,
(R)-2-[3-(4-methoxyphenyl)ureido]-3-phenylpropionic acid,
(R)-2-[3-(4-bromophenyl)ureido]-3-phenylpropionic acid,
(R)-2-[3-(4-iodophenyl)ureido]-3-phenylpropionic acid,
(R)-2-[3-(4-fluorophenyl)ureido]-3-phenylpropionic acid,
(S)-2-[3-(3-trifluorophenyl)ureido]-3-phenylpropionic acid,
(R)-2-[3-(3-trifluorophenyl)ureido]-3-phenylpropionic acid, gives the following compounds:
(S)-2-(3-pyridin-2-ylureido)-N-(2′-methylsulfonylbiphenyl-4-yl)-pentanoamide, ESI 467; IC₅₀ (Xa)=3.8×10⁻⁶ M; IC₅₀ (VIIa)=2.7×10⁻⁶ M;
(R)-2-(3-phenylureido)-N-(2′-methylsulfonylbiphenyl-4-yl)pentanoamide, ESI 466; IC₅₀ (Xa)=2×10⁻⁶ M; Ic₅₀ (VIIa)=9.3×10⁻⁷ M;
2-(3-phenylureido)-N-(2′-methylsulfonylbiphenyl-4-yl)-3-(thiophen-2-yl)-propionamide, ESI 520; IC₅₀ (Xa)=1.2×10⁻⁶ M; IC₅₀ (VIIa)=7.5×10⁻⁷ M;
2-(3-phenylureido)-N-(2′-methylsulfonylbiphenyl-4-yl)-3-(3H-imidazol-4-yl)propionamide, ESI 504; IC₅₀ (Xa)=2×10⁻⁶ M; IC₅₀ (VIIa)=2×10⁻⁶ M;
(R)-2-(3-phenylureido)-N-(2′-methylsulfonylbiphenyl-4-yl)hexanoamide, ESI 480; IC₅₀ (Xa)=3×10⁻⁶ M; IC₅₀ (VIIa)=1.7×10⁻⁷ M;
2-(3-phenylureido)-N-(2′-methylsulfonylbiphenyl-4-yl)-4-methylsulfanylbutyramide, ESI 498; IC₅₀ (Xa)=2.3×10⁻⁶ M; IC₅₀ (VIIa)=1.8×10⁻⁶ M;
2-(3-phenylureido)-N-(2′-methylsulfonylbiphenyl-4-yl )-2-phenyl-acetamide, ESI 500; IC₅₀ (Xa)=2.3×10⁻⁶ M; IC₅₀ (VIIa)=2×10⁻⁶ M;
(S)-2-[3-(4-chlorophenyl)ureido]-N-(2′-methylsulfonylbiphenyl-4-yl)-3-phenylpropionamide, ESI 548;
(R)-2-[3-(4-methylphenyl)ureido]-N-(2′-methylsulfonylbiphenyl-4-yl)-3-phenylpropionamide, ESI 528;
(R)-2-(3-pyridin-4-ylureido)-N-(2′-methylsulfonylbiphenyl-4-yl)pentanoamide, ESI 467;
(S)-2-(3-pyridin-4-ylureido)-N-(2′-methylsulfonylbiphenyl-4-yl)pentanoamide, ESI 467;
(R)-2-(3-pyridin-2-ylureido)-N-(2′-methylsulfonylbiphenyl-4-yl)pentanoamide, ESI 467;
(S)-2-(3-phenylureido)-N-(2′-methylsulfonylbiphenyl-4-yl)pentanoamide, ESI 466;
(R)-2-(3-pyridin-3-ylureido)-N-(2′-methylsulfonylbiphenyl-4-yl)-pentanoamide, ESI 467;
(S)-2-(3-phenylureido)-N-(2′-methylsulfonylbiphenyl-4-yl)-3-(pyridin-3-yl)propionamide, ESI 515;
(S)-2-(3-phenylureido)-N-(2′-methylsulfonylbiphenyl-4-yl)-3-(indol-3-yl)-propionamide, ESI 553;
2-(3-phenylureido)-N-(2′-methylsulfonylbiphenyl-4-yl)propionamide, ESI 438;
2-(3-phenylureido)-N-(2′-methylsulfonylbiphenyl-4-yl)acetamide, ESI 424;
(S)-2-[3-(3-chlorophenyl)ureido]-N-(2′-methylsulfonylbiphenyl-4-yl )-3-phenylpropionamide, ESI 548;
(S)-2-[3-(4-trifluoromethylphenyl)ureido]-N-(2′-methylsulfonylbiphenyl-4-yl)-3-phenylpropionamide, ESI 582;
(S)-2-[3-(2-chlorophenyl)ureido]-N-(2′-methylsulfonylbiphenyl-4-yl )-3-phenylpropionamide, ESI 548;
(S)-2-[3-(4-ethoxyphenyl)ureido]-N-(2′-methylsulfonylbiphenyl-4-yl)-3-phenylpropionamide, ESI 558;
(S)-2-[3-(4-methylphenyl)ureido]-N-(2′-methylsulfonylbiphenyl-4-yl)-3-phenylpropionamide, ESI 528;
(S)-2-[3-(2-methoxyphenyl)ureido]-N-(2′-methylsulfonylbiphenyl-4-yl )-3-phenylpropionamide, ESI 544;
(S)-2-[3-(4-ethoxycarbonylphenyl)ureido]-N-(2′-methylsulfonylbiphenyl-4-yl)-3-phenylpropionamide, ESI 586;
(R)-2-[3-(3-chlorophenyl)ureido]-N-(2′-methylsulfonylbiphenyl-4-yl)-3-phenylpropionamide, ESI 548;
(R)-2-[3-(4-trifluoromethylphenyl)ureido]-N-(2′-methylsulfonylbiphenyl-4-yl)-3-phenylpropionamide, ESI 582;
(R)-2-[3-(2-chlorophenyl)ureido]-N-(2′-methylsulfonylbiphenyl-4-yl)-3-phenylpropionamide, ESI 548;
(R)-2-[3-(4-ethoxyphenyl)ureido]-N-(2′-methylsulfonylbiphenyl-4-yl)-3-phenylpropionamide, ESI 558;
(R)-2-[3-(2-methoxyphenyl)ureido]-N-(2′-methylsulfonylbiphenyl-4-yl)-3-phenylpropionamide, ESI 544;
(R)-2-[3-(4-ethoxycarbonylphenyl)ureido]-N-(2′-methylsulfonylbiphenyl-4-yl)-3-phenylpropionamide, ESI 586;
2-(3-phenylureido)-N-(2′-methylsulfonylbiphenyl-4-yl)-5-BOC-amino-valeramide,
(S)-2-(3-phenylureido)-N-(2′-methylsulfonylbiphenyl-4-yl)-3-phenylpropionamide, ESI 514;
(R)-2-(3-phenylureido)-N-(2′-methylsulfonylbiphenyl-4-yl)-3-phenylpropionamide, ESI 514;
(R)-2-[3-(4-chlorophenyl)ureido]-N-(2′-methylsulfonylbiphenyl-4-yl)-3-cyclopropylpropionamide;
2-[3-(4-chlorophenyl)ureido]-N-(2′-methylsulfonylbiphenyl-4-yl)-4-methylsulfanylbutyramide, ESI 532;
(R)-2-[3-(4-chlorophenyl)ureido]-N-(2′-methylsulfonylbiphenyl-4-yl)-propionamide, ESI 472;
2-[3-(4-chlorophenyl)ureido]-N-(2′-methylsulfonylbiphenyl-4-yl)acetamide, ESI 458;
2-[3-(5-chloropyridin-2-yl)ureido]-N-(2′-methylsulfonylbiphenyl-4-yl)-3-phenylpropionamide;
(R)-2-[3-(4-bromophenyl)ureido]-N-(2′-methylsulfonylbiphenyl-4-yl )-3-phenylpropionamide;
(R)-2-[3-(3-fluoro-4-methoxyphenyl)ureido]-N-(2′-methylsulfonyl-biphenyl-4-yl)-3-phenylpropionamide, ESI 562;
2-[3-(4-chlorophenyl)ureido]-N-(2′-methylsulfonylbiphenyl-4-yl)hexanoamide, ESI 514;
(R)-2-[3-(4-chlorophenyl)ureido]-N-(2′-methylsulfonylbiphenyl-4-yl)-2-phenylacetamide, ESI 534;
(S)-2-[3-(4-chlorophenyl)ureido]-N-(2′-methylsulfonylbiphenyl-4-yl )-4-methylpentanoamide,
(R)-2-[3-(4-chlorophenyl)ureido]-N-(2′-methylsulfonylbiphenyl-4-yl)-4-methylpentanoamide,
(S)-2-[3-(4-methoxyphenyl)ureido]-N-(2′-methylsulfonylbiphenyl-4-yl)-3-phenylpropionamide, ESI 544;
(S)-2-[3-(4-bromophenyl)ureido]-N-(2′-methylsulfonylbiphenyl-4-yl)-3-phenylpropionamide,
(S)-2-[3-(4-fluorophenyl)ureido]-N-(2′-methylsulfonylbiphenyl-4-yl)-3-phenylpropionamide, ESI 640;
(S)-2-[3-(4-fluorophenyl)ureido]-N-(2′-methylsulfonylbiphenyl-4-yl)-3-phenylpropionamide, ESI 532;
(S)-2-[3-(3-fluoro-4-methoxyphenyl)ureido]-N-(2′-methylsulfonylbiphenyl-4-yl)-3-phenylpropionamide,
(R)-2-[3-(4-methoxyphenyl)ureido]-N-(2′-methylsulfonylbiphenyl-4-yl)-3-phenylpropionamide, ESI 544;
(R)-2-[3-(4-bromophenyl)ureido]-N-(2′-methylsulfonylbiphenyl-4-yl)-3-phenylpropionamide,
(R)-2-[3-(4-fluorophenyl)ureido]-N-(2′-methylsulfonylbiphenyl-4-yl)-3-phenylpropionamide, ESI 640;
(R)-2-[3-(4-iodophenyl)ureido]-N-(2′-methylsulfonylbiphenyl-4-yl)-3-phenylpropionamide, ESI 532;
(S)-2-[3-(3-trifluoromethylphenyl)ureido]-N-(2′-methylsulfonylbiphenyl-4-yl)-3-phenylpropionamide, ESI 582;
(R)-2-[3-(3-trifluoromethylphenyl)ureido]-N-(2′-methylsulfonylbiphenyl-4-yl)-3-phenylpropionamide, ESI 582.

### EXAMPLE 2a

Removal of the BOC protecting group from 2-(3-phenylureido)-N-(2′-methylsulfonylbiphenyl-4-yl)-5-BOC-aminovaleramide gives the compound 2-(3-phenylureido)-N-(2′-methylsulfonylbiphenyl-4-yl)-5-aminovaleramide, hydrochloride, ESI 481.

### EXAMPLE 3

Analogous reaction of 4-(morpholin-4-yl)aniline with
(S)-2-(3-phenylureido)-3-phenylpropionic acid,
2-(3-phenylureido)valeric acid,
(R)-2-(3-phenylureido)-3-phenylpropionic acid,
2-(3-phenylureido)-3-(3-cyanophenyl)propionic acid,
2-[3-(4-chlorophenyl)ureido]caproic acid,
2-[3-(4-chlorophenyl)ureido]-4-(methylsulfanyl)butyric acid,
(R)-2-[3-(4-chlorophenyl)ureido]propionic acid,
(S)-2-[3-(4-chlorophenyl)ureido]-4-methylvaleric acid,
(R)-2-[3-(4-chlorophenyl)ureido]-4-methylvaleric acid,
   gives the following compounds:
(S)-2-(3-phenylureido)-N-[4-(morpholin-4-yl)phenyl]-3-phenylpropionamide, ESI 445 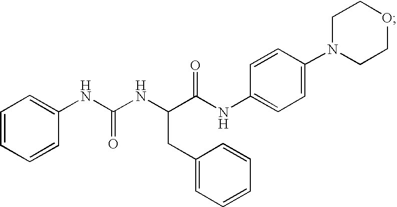
2-(3-phenylureido)-N-[4-(morpholin-4-yl)phenyl]valeramide, ESI 397;
(R)-2-(3-phenylureido)-N-[4-(morpholin-4-yl)phenyl]-3-phenylpropion-amide, ESI 445;
2-(3-phenylureido)-N-[4-(morpholin-4-yl)-phenyl]-3-(3-cyanophenyl)-propionamide, ESI 470;
2-[3-(4-chlorophenyl)ureido]-N-[4-(morpholin-4-yl)phenyl]caproamide, ESI 445;
2-[3-(4-chlorophenyl)ureido]-N-[4-(morpholin-4-yl)phenyl]-4-methyl-sulfanylbutyramide, ESI 463;
(R)-2-[3-(4-chlorophenyl)ureido]-N-[4-(morpholin-4-yl)phenyl]propion-amide, ESI 403;
(S)-2-[3-(4-chlorophenyl)ureido]-N-[4-(morpholin-4-yl)phenyl]-4-methylvaleramide, ESI 445;
(R)-2-[3-(4-chlorophenyl)ureido]-N-[4-(morpholin-4-yl)phenyl]-4-methylvaleramide, ESI 445.

### EXAMPLE 4

Reaction of 1-(pyridin-4-yl)piperidin-4-ylmethylamine with
(S)-2-(3-phenylureido)-3-phenylpropionic acid,
(R)-2-(3-phenylureido)-3-phenylpropionic acid,
2-(3-phenylureido)valeric acid,
(S)-2-[3-(4-chlorophenyl)ureido]-2-phenylacetic acid,
2-[3-(4-chlorophenyl)ureido]caproic acid,
2-[3-(4-chlorophenyl)ureido]-4-(methylsulfanyl)butyric acid,
(R)-2-[3-(4-chlorophenyl)ureido]propionic acid,
2-[3-(4-chlorophenyl)ureido]-3-(thiophen-2-yl )propionic acid,
2-[3-(4-chlorophenyl)ureido]-3-(indol-3-yl)propionic acid,
2-[3-(4-chlorophenyl)ureido]valeric acid,
(S)-2-[3-(4-chlorophenyl)ureido]-4-methylvaleric acid,
(R)-2-[3-(4-chlorophenyl)ureido]-4-methylvaleric acid,
(R)-2-[3-(4-chlorophenyl)ureido]-2-phenylacetic acid,
(R)-2-[3-(4-chlorophenyl)ureido]-4-methylbutyric acid,
(S)-2-[3-(4-chlorophenyl)ureido]-4-methylbutyric acid,
(R)-2-[3-(3-chloro-pyridin-6-yl)ureido]-2-phenylacetic acid,
2-[3-(4-chlorophenyl)ureido]-3,3,3-trifluoropropionic acid,
2-[3-(4-chlorophenyl)ureido]-2-(pyridin-2-yl)acetic acid,
(R)-2-[3-(4-chlorophenyl)ureido]-2-(tert-butyl)acetic acid,
(S)-2-[3-(4-chlorophenyl)ureido]-2-(tert-butyl)acetic acid,
2-[3-(4-chlorophenyl)ureido]-2-(2-fluorophenyl)acetic acid,
(R)-2-[3-(4-chlorophenyl)ureido]-2-(4-fluorophenyl)acetic acid,
(S)-2-[3-(4-chlorophenyl)ureido]-2-(4-fluorophenyl)acetic acid,
(R)-2-[3-(4-chlorophenyl)ureido]-2-(4-hydroxyphenyl)acetic acid,
(S)-2-[3-(4-chlorophenyl)ureido]-2-(4-hydroxyphenyl)acetic acid,
2-[3-(4-chlorophenyl)ureido]acetic acid,
(S)-2-[3-(4-chlorophenyl)ureido]-3-phenylpropionic acid,
(R)-2-[3-(4-chlorophenyl)ureido]-3-phenylpropionic acid,
2-[3-(4-chlorophenyl)ureido]-2-(2,1,3-benzothiadiazol-5-yl)acetic acid,
   analogously to Example 2 gives the following compounds:
(S)-2-(3-phenylureido)-N-[1-(pyridin-4-yl)piperidin-4-ylmethyl]-3-phenylpropionamide, ESI 458 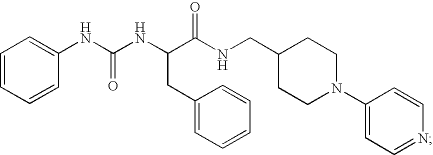
(R)-2-(3-phenylureido)-N-[1-(pyridin-4-yl)piperidin-4-ylmethyl]-3-phenylpropionamide, ESI 458;
2-(3-phenylureido)-N-[1-(pyridin-4-yl)piperidin-4-ylmethyl]valeramide, ESI 410;
(S)-2-[3-(4-chlorophenyl)ureido]-N-[1-(pyridin-4-yl)piperidin-4-ylmethyl]-2-phenylacetamide, ESI 478;
(R)-2-[3-(4-chlorophenyl)ureido]-N-[1-(pyridin-4-yl)piperidin-4-yl-methyl]caproamide, ESI 458;
2-[3-(4-chlorophenyl)ureido]-N-[1-(pyridin-4-yl)piperidin-4-ylmethyl]-4-methylsulfanylbutyramide, ESI 476;
(R)-2-[3-(4-chlorophenyl)ureido]-N-[1-(pyridin-4-yl)piperidin-4-yl-methyl]propionamide, ESI 416;
2-[3-(4-chlorophenyl)ureido]-N-[1-(pyridin-4-yl)piperidin-4-ylmethyl]-3-(thiophen-2-yl)propionamide, ESI 498;
2-[3-(4-chlorophenyl)ureido]-N-[1-(pyridin-4-yl)piperidin-4-ylmethyl]-3-(indol-3-yl)propionamide, ESI 531;
2-[3-(4-chlorophenyl)ureido]-N-[1-(pyridin-4-yl)piperidin-4-ylmethyl]-valeramide, ESI 444; IC₅₀ (Xa)=5.8×10⁻⁷ M;
(S)-2-[3-(4-chlorophenyl)ureido]-N-[1-(pyridin-4-yl)piperidin-4-ylmethyl]-4-methylvaleramide, ESI 459;
(R)-2-[3-(4-chlorophenyl)ureido]-N-[1-(pyridin-4-yl)piperid in-4-ylmethyl]-4-methylvaleramide, ESI 459; IC₅₀ (Xa)=4.1×10⁻⁷ M;
(R)-2-[3-(4-chlorophenyl)ureido]-N-[1-(pyridin-4-yl)piperidin-4-ylmethyl]-2-phenylacetamide, ESI 478; IC₅₀ (Xa)=5.5×10⁻⁸ M;
(R)-2-[3-(4-chlorophenyl)ureido]-N-[1-(pyridin-4-yl)piperidin-4-ylmethyl]-4-methylbutyramide, ESI 444;
(S)-2-[3-(4-chlorophenyl)ureido]-N-[1-(pyridin-4-yl)piperidin-4-ylmethyl]-4-methylbutyramide, ESI 444;
(R)-2-[3-(3-chloropyridin-6-yl)ureido]-N-[1-(pyrid in-4-yl)piperid in-4-yl-methyl]-2-phenylacetamide,
2-[3-(4-chlorophenyl)ureido]-N-[1-(pyridin4-yl)piperidin-4-ylmethyl]-3,3,3-trifluoropropionamide,
2-[3-(4-chlorophenyl)ureido]-N-[1-(pyridin-4-yl)piperidin-4-ylmethyl]-2-(pyridin-2-yl)acetamide, ESI 479;
(R)-2-[3-(4-chlorophenyl)ureido]-N-[1-(pyridin-4-yl)piperidin-4-ylmethyl]-2-(tert-butyl)acetamide, ESI 458;
(S)-2-[3-(4-chlorophenyl)ureido]-N-[1-(pyridin-4-yl)piperidin-4-ylmethyl]-2-(tert-butyl)acetamide,
2-[3-(4-chlorophenyl)ureido]-N-[1-(pyridin-4-yl)piperidin-4-ylmethyl]-2-(2-fluorophenyl)acetamide, ESI 496;
(R)-2-[3-(4-chlorophenyl)ureido]-N-[1-(pyridin-4-yl)piperidin-4-ylmethyl]-2-(4-fluorophenyl)acetamide, ESI 496;
(S)-2-[3-(4-chlorophenyl)ureido]-N-[1-(pyridin-4-yl)piperidin-4-ylmethyl]-2-(4-fluorophenyl)acetamide,
(R)-2-[3-(4-chlorophenyl)ureido]-N-[1-(pyridin-4-yl )piperidin-4-ylmethyl]-2-(4-hydroxyphenyl)acetamide, ESI 494;
(S)-2-[3-(4-chlorophenyl)ureido]-N-[1-(pyridin-4-yl)piperidin-4-ylmethyl]-2-(4-hydroxyphenyl)acetamide,
2-[3-(4-chlorophenyl)ureido]-N-[1-(pyridin-4-yl)piperidin-4-ylmethyl]-acetamide, ESI 402;
(S)-2-[3-(4-chlorophenyl)ureido]-N-[1-(pyridin-4-yl)piperidin-4-ylmethyl]-3-phenylpropionamide, ESI 492;
(R)-2-[3-(4-chlorophenyl)ureido]-N-[1-(pyridin-4-yl)piperid in-4-ylmethyl]-3-phenylpropionamide, ESI 492;
2-[3-(3-chloropyridin-6-yl)ureido]-N-[1-(pyrid in-4-yl)piperidin-4-yl-methyl]-2-(2,1,3-benzothiadiazol-5-yl)acetamide, ESI 536.

### EXAMPLE 5

Reaction of C-biphenyl-2yl-methylamine with
(S)-2-(3-phenylureido)-3-phenylpropionic acid,
(R)-2-(3-phenylureido)-3-phenylpropionic acid,
2-(3-phenylureido)valeric acid,
   analogously to Example 2 gives the following compounds:
(S)-2-(3-phenylureido)-N-(biphenyl-2-ylmethyl)-3-phenyl-propionamide, ESI 450;
(R)-2-(3-phenylureido)-N-(biphenyl-2-ylmethyl)-3-phenyl-propionamide, ESI 450;
2-(3-phenylureido)-N-(biphenyl-2-ylmethyl)valeramide, ESI 402.

### EXAMPLE 6

Reaction of 2′-methylsulfonylbiphenyl-4-yl-methylamine analogously to Example 2 with
(S)-2-(3-phenylureido)-3-phenylpropionic acid,
(R)-2-(3-phenylureido)-3-phenylpropionic acid,
2-(3-phenylureido)valeric acid,
   gives the following compounds:
(S)-2-(3-phenylureido)-N-(2′-methylsulfonylbiphenyl-4-ylmethyl)-3-phenylpropionamide, ESI 528;
(R)-2-(3-phenylureido)-N-(2′-methylsulfonylbiphenyl-4-ylmethyl)-3-phenylpropionamide, ESI 528;
2-(3-phenylureido)-N-(2′-methylsulfonylbiphenyl-4-ylmethyl)valeramide, ESI 480.

### EXAMPLE 7

Reaction of 1-(pyridin-4-yl)piperidin-4-ylamine analogously to Example 2 with
(R)-2-[3-(4-chlorophenyl)ureido]-3-phenylpropionic acid,
(R)-2-[3-(4-chlorophenyl)ureido]-2-phenylacetic acid,
2-[3-(4-chlorophenyl)ureido]pentanoic acid,
(S)-2-[3-(4-chlorophenyl)ureido]-3-phenylpropionic acid,
   gives the following compounds:
(R)-2-[3-(4-chlorophenyl)ureido]-N-[1-(pyridin-4-yl)-piperidin-4-yl]-3-phenylpropionamide;
(R)-2-[3-(4-chlorophenyl)ureido]-N-[1-(pyridin-4-yl)-piperidin-4-yl]-2-phenylacetamide, hydrochloride, ESI 464;
2-[3-(4-chlorophenyl)ureido]-N-[1-(pyridin-4-yl)-piperidin-4-yl]-pentanoamide, ESI 430;
(S)-2-[3-(4-chlorophenyl)ureido]-N-[1-(pyridin-4-yl)-piperidin-4-yl]-3-phenylpropionamide, hydrochloride, ESI 478.

### EXAMPLE 8

Reaction of 2′-tert-butylaminosulfonylbiphenyl-4-ylamine analogously to Example 2 with
(R)-2-[3-(4-chlorophenyl)ureido]-3-phenylpropionic acid,
   gives the following compound:
(R)-2-[3-(4-chlorophenyl)ureido]-N-(2′-tert-butylaminosulfonyl-biphenyl-4-yl)-3-phenylpropionamide,
   and removal of the protecting group gives the compound
(R)-2-[3-(4-chlorophenyl)ureido]-N-(2′-aminosulfonylbiphenyl-4-yl)-3-phenylpropionamide.

### EXAMPLE 9

Reaction of 1-(tetrahydropyran-4-yl)piperidin-4-ylamine analogously to Example 2 with
(R)-2-[3-(4-chlorophenyl)ureido]-3-phenylpropionic acid
   gives the following compound:
(R)-2-[3-(4-chlorophenyl)ureido]-N-[1-(tetrahydropyran-4-yl)-piperidin-4-yl]-3-phenylpropionamide.

### EXAMPLE 10

Reaction of 1-isopropylpiperidin-4-ylamine with
(R)-2-[3-(4-chlorophenyl)ureido]-3-phenylpropionic acid,
(S)-2-[3-(4-chlorophenyl)ureido]-3-phenylpropionic acid,
2-[3-(4-chlorophenyl)ureido]valeric acid,
(S)-2-[3-(4-chlorophenyl)ureido]-2-phenylacetic acid,
   analogously to Example 2 gives the following compounds:
(R)-2-[3-(4-chlorophenyl)ureido]-N-[1-isopropylpiperidin-4-yl]-3-phenylpropionamide, hydrochloride;
(S)-2-[3-(4-chlorophenyl)ureido]-N-[1-isopropylpiperidin-4-yl]-3-phenylpropionamide, hydrochloride, ESI 443;
2-[3-(4-chlorophenyl)ureido]-N-[1-isopropylpiperidin-4-yl]valeramide, hydrochloride, ESI 395;
(S)-2-[3-(4-chlorophenyl)ureido]-N-[1-isopropylpiperidin-4-yl]-2-phenyl-acetamide, hydrochloride, ESI 429.

### EXAMPLE 11

Reaction of 1-(tetrahydropyran-4-yl )-piperidin-4-ylmethylamine with
(R)-2-[3-(4-chlorophenyl)ureido]-3-phenylpropionic acid,
(R)-2-[3-(4-chlorophenyl)ureido]-2-phenylacetic acid,
   analogously to Example 2 gives the following compounds:
(R)-2-[3-(4-chlorophenyl)ureido]-N-[1-(tetrahydropyran-4-yl)-piperidin-4-ylmethyl]-3-phenylpropionamide;
(R)-2-[3-(4-chlorophenyl)ureido]-N-[1-(tetrahydropyran-4-yl)-piperidin-4-ylmethyl]-2-phenylacetamide, ESI 471.

### EXAMPLE 12

Reaction of 4-(2-oxopiperidin-1-yl)aniline with
(R)-2-[3-(4-chlorophenyl)ureido]-3-phenylpropionic acid,
(S)-2-[3-(4-chlorophenyl)ureido]-3-phenylpropionic acid,
   analogously to Example 2 gives the following compound:
(R)-2-[3-(4-chlorophenyl)ureido]-N-[4-(2-oxopiperidin-1-yl)phenyl]-3-phenylpropionamide, ESI 491,
(S)-2-[3-(4-chlorophenyl)ureido]-N-[4-(2-oxopiperidin-1-yl)phenyl]-3-phenylpropionamide, ESI 491.

### EXAMPLE 13

Reaction of 4-(3-oxomorpholin-4-yl)phenylamine with
(R)-2-[3-(4-chlorophenyl)ureido]-3-phenylpropionic acid
   analogously to Example 2 gives the following compound:
(R)-2-[3-(4-chlorophenyl)ureido]-N-[4-(3-oxomorpholin-4-yl)phenyl]-3-phenylpropionamide.

### EXAMPLE 14

14.1 2.0 g of chlorophenyl isocyanate and 100 mg of dibutyltin dilaurate are added to a solution of 2.0 g of D/L-mandelic acid in 20 ml of dichloromethane, and the mixture is stirred at room temperature for 18 hours. Conventional work-up gives 2-[N-(4-chlorophenyl)carbamoyloxy]-2-phenylacetic acid (“CA”), ESI 306.

14.2 36 ml of 4-methylmorpholine are added to a solution of 100 mg of “CA”, 63 mg of 1-(pyridin-4-yl)piperidin-4-ylmethylamine, 63 mg of DAPECI and 45 mg of HOBt in 2 ml of DMF, and the mixture is stirred at room temperature for 18 hours. Conventional work-up gives 2-[N-(4-chlorophenyl)-carbamoyloxy]-N-[1-(pyridin-4-yl)piperidin-4-ylmethyl]-2-phenylacetamide, ESI 479, 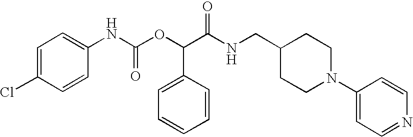
IC₅₀ (Xa)=7.1×10⁻⁸ M.

The following compounds are obtained analogously starting from (R)- and (S)-mandelic acid:
(S)-2-[N-(4-chlorophenyl)carbamoyloxy]-N-[1-(pyridin-4-yl)-piperidin-4-ylmethyl]-2-phenylacetamide, hydrochloride, ESI 479 and
(R)-2-[N-(4-chlorophenyl)carbamoyloxy]-N-[1-(pyridin-4-yl)-piperidin-4-ylmethyl]-2-phenylacetamide, hydrochloride, ESI 479,
   Analogous reaction of 1-(pyridin-4-yl)piperidin-4-ylmethylamine with
2-[N-(4-chlorophenyl)carbamoyloxy]acetic acid,
2-[N-(4-chlorophenyl)carbamoyloxy]propionic acid,
2-[N-(4-chlorophenyl)carbamoyloxy]-2-(2-fluorophenyl)acetic acid,
2-[N-(4-chlorophenyl)carbamoyloxy]-2-(4-chlorophenyl)acetic acid,
2-[N-(4-chlorophenyl)carbamoyloxy]-2-(2-chlorophenyl)acetic acid,
(R)-2-[N-(4-chlorophenyl)carbamoyloxy]-2-(3-chlorophenyl)acetic acid,
   gives the following compounds:
2-[N-(4-chlorophenyl)carbamoyloxy]-N-[1-(pyridin-4-yl)-piperidin-4-yl-methyl]acetamide, ESI 403;
2-[N-(4-chlorophenyl)carbamoyloxy]-N-[1-(pyridin-4-yl)-piperidin-4-yl-methyl]propionamide, ESI 417.
2-[N-(4-chlorophenyl)carbamoyloxy]-2-(2-fluorophenyl)-N-[1-(pyridin-4-yl)-piperidin-4-ylmethyl]acetamide, ESI 497;
2-[N-(4-chlorophenyl)carbamoyloxy]-2-(4-chlorophenyl)-N-[1-(pyrid in-4-yl)-piperidin-4-ylmethyl]acetamide, ESI 513;
2-[N-(4-chlorophenyl)carbamoyloxy]-2-(2-chlorophenyl)-N-[1-(pyridin-4-yl)-piperidin-4-ylmethyl]acetamide, ESI 513;
(R)-2-[N-(4-chlorophenyl)carbamoyloxy]-2-(3-chlorophenyl)-N-[1-(pyridin-4-yl)-piperidin-4-ylmethyl]acetamide, ESI 513;

### EXAMPLE 15

Reaction of 1-cyclopentylpiperidin-4-ylamine with
(S)-2-[3-(4-chlorophenyl)ureido]-3-phenylpropionic acid,
(R)-2-[3-(4-chlorophenyl)ureido]-3-phenylpropionic acid,
(R)-2-[3-(4-chlorophenyl)ureido]-2-phenylacetic acid,
   analogously to Example 2 gives the following compounds:
(S)-2-[3-(4-chlorophenyl)ureido]-N-[1-cyclopentylpiperidin-4-yl]-3-phenylpropionamide, ESI 469;
(R)-2-[3-(4-chlorophenyl)ureido]-N-[1-cyclopentylpiperidin-4-yl]-3-phenylpropionamide, ESI 469;
(R)-2-[3-(4-chlorophenyl)ureido]-N-[1-cyclopentylpiperidin-4-yl]-2-phenylacetamide, ESI 455.

### EXAMPLE 16

Reaction of 4-(2-oxopyrrolidin-1-yl)aniline with
(R)-2-[3-(4-chlorophenyl)ureido]-3-phenylpropionic acid,
(S)-2-[3-(4-chlorophenyl)ureido]-3-phenylpropionic acid,
   analogously to Example 2 gives the following compounds:
(R)-2-[3-(4-chlorophenyl)ureido]-N-[4-(2-oxopyrrolidin-1-yl)phenyl]-3-phenylpropionamide, ESI 477,
(S)-2-[3-(4-chlorophenyl)ureido]-N-[4-(2-oxopyrrolidin-1-yl)phenyl]-3-phenylpropionamide.

### EXAMPLE 17

Reaction of 4-(piperidin-1-yl)aniline with
(R)-2-[3-(4-chlorophenyl)ureido]-3-phenylpropionic acid,
(S)-2-[3-(4-chlorophenyl)ureido]-3-phenylpropionic acid,
(R)-2-[3-(4-chlorophenyl)ureido]-2-phenylacetic acid,
   analogously to Example 2 gives the following compounds:
(R)-2-[3-(4-chlorophenyl)ureido]-N-[4-(piperidin-1-yl)-phenyl]-3-phenylpropionamide, ESI 477,
(S)-2-[3-(4-chlorophenyl)ureido]-N-[4-(piperidin-1-yl)-phenyl]-3-phenylpropionamide, ESI 477;
(R)-2-[3-(4-chlorophenyl)ureido]-N-[4-(piperidin-1-yl)-phenyl]-2-phenylacetamide, ESI 463.

### EXAMPLE 18

Reaction of 4-diethylaminoaniline with
(R)-2-[3-(4-chlorophenyl)ureido]-3-phenylpropionic acid,
(S)-2-[3-(4-chlorophenyl)ureido]-3-phenylpropionic acid,
(R)-2-[3-(4-chlorophenyl)ureido]-2-phenylacetic acid,
   analogously to Example 2 gives the following compounds:
(R)-2-[3-(4-chlorophenyl)ureido]-N-[4-diethylaminophenyl]-3-phenylpropionamide, ESI 465;
(S)-2-[3-(4-chlorophenyl)ureido]-N-[4-diethylaminophenyl]-3-phenylpropionamide, ESI 465;
(R)-2-[3-(4-chlorophenyl)ureido]-N-[4-diethylaminophenyl]-2-phenylacetamide, ESI 451.

The compounds
(R)-2-[3-(4-chlorophenyl)ureido]-N-[4-dimethylaminophenyl]-3-phenylpropionamide, ESI 437;
(S)-2-[3-(4-chlorophenyl)ureido]-N-[4-dimethylaminophenyl]-3-phenylpropionamide, ESI 437,
   are obtained analogously.

### EXAMPLE 19

Reaction of 1-(tetrahydropyran-4-yl)piperidin-4-ylamine with
(R)-2-[3-(4-chlorophenyl)ureido]-3-phenylpropionic acid
   analogously to Example 2 gives the following compound:
(R)-2-[3-(4-chlorophenyl)ureido]-N-[1-(tetrahydropyran-4-yl)-piperidin-4-yl]-3-phenylpropionamide, ESI 485.

### EXAMPLE 20

Reaction of 4-aminomethyl-1-BOC-piperidine with
(S)-2-[3-(4-chlorophenyl)ureido]-3-phenylpropionic acid,
(R)-2-[3-(4-chlorophenyl)ureido]-3-phenylpropionic acid,
(S)-2-[3-(4-chlorophenyl)ureido]-4-methylpentanoic acid,
(R)-2-[3-(4-chlorophenyl)ureido]-4-methylpentanoic acid,
(R)-2-[3-(4-chlorophenyl)ureido]-2-phenylacetic acid,
   analogously to Example 2 gives the following compounds:
(S)-2-[3-(4-chlorophenyl)ureido]-N-(1-BOC-piperidin-4-ylmethyl)-3-phenylpropionamide,
(R)-2-[3-(4-chlorophenyl)ureido]-N-(1-BOC-piperidin-4-ylmethyl)-3-phenylpropionamide,
(S)-2-[3-(4-chlorophenyl)ureido]-N-(1-BOC-piperidin-4-ylmethyl)-4-methylpentanoamide,
(R)-2-[3-(4-chlorophenyl)ureido]-N-(1-BOC-piperidin-4-ylmethyl)-4-methylpentanoamide,
(R)-2-[3-(4-chlorophenyl)ureido]-N-(1-BOC-piperidin-4-ylmethyl)-2-phenylacetamide, ESI 501.

### EXAMPLE 20a

Removal of the BOC protecting group from the compounds obtained in Example 20 using HCl in dioxane gives the following piperidine derivatives
(S)-2-[3-(4-chlorophenyl)ureido]-N-(piperidin-4-ylmethyl)-3-phenylpropionamide, hydrochloride, ESI 415;
(R)-2-[3-(4-chlorophenyl)ureido]-N-(piperidin-4-ylmethyl)-3-phenylpropionamide, hydrochloride, ESI 415;
(S)-2-[3-(4-chlorophenyl)ureido]-N-(piperidin-4-ylmethyl)-4-methyl-pentanoamide, hydrochloride, ESI 381;
(R)-2-[3-(4-chlorophenyl)ureido]-N-(piperidin-4-ylmethyl)-4-methyl-pentanoamide, hydrochloride, ESI 381;
(R)-2-[3-(4-chlorophenyl)ureido]-N-(piperidin-4-ylmethyl)-2-phenylacetamide, hydrochloride, ESI 401.

### EXAMPLE 21

Reaction of (1-isopropylpiperidin-4-yl)methylamine with
(S)-2-[3-(4-chlorophenyl)ureido]-3-phenylpropionic acid,
(R)-2-[3-(4-chlorophenyl)ureido]-3-phenylpropionic acid,
(S)-2-[3-(4-chlorophenyl)ureido]-4-methylpentanoic acid,
(R)-2-[3-(4-chlorophenyl)ureido]-4-methylpentanoic acid,
(R)-2-[3-(4-chlorophenyl)ureido]-2-phenylacetic acid,
   analogously to Example 2 gives the following compounds:

(S)-2-[3-(4-chlorophenyl)ureido]-N-[1-isopropylpiperidin-4-yl]-3-phenylpropionamide, ESI 457;
(R)-2-[3-(4-chlorophenyl)ureido]-N-[1-isopropylpiperidin-4-yl]-3-phenylpropionamide, ESI 457;
(S)-2-[3-(4-chlorophenyl)ureido]-N-[1-isopropylpiperidin-4-yl]-4-methylpentanoamide, ESI 423;
(R)-2-[3-(4-chlorophenyl)ureido]-N-[1-isopropylpiperidin-4-yl]-4-methylpentanoamide, ESI 423;
(R)-2-[3-(4-chlorophenyl)ureido]-N-[1-isopropylpiperidin-4-yl]-2-phenylacetamide, ESI 443.

### EXAMPLE 21a

Partitioning of (R)-2-[3-(4-chlorophenyl)ureido]-N-(piperidin-4-ylmethyl)-2-phenylacetamide, hydrochloride, between ethyl acetate and 1 N NaOH, followed by removal of the solvent, gives the free base.

120 mg of (R)-2-[3-(4-chlorophenyl)ureido]-N-(piperidin-4-ylmethyl)-2-phenylacetamide is dissolved in 4 ml of dichloromethane and 2 ml of acetone, and 0.1 ml of acetic acid and 300 mg of sodium triacetoxyborohydride are added, and the mixture is stirred at room temperature for 18 hours. Saturated aqueous ammonium chloride solution is then added, and the organic phase is separated off. Removal of the solvent gives (R)-2-[3-(4-chlorophenyl)ureido]-N-[1-isopropylpiperidin-4-yl]-2-phenylacetamide, ES I 443.

### EXAMPLE 22

Reaction of 4-(4-BOC-piperazin-1-yl)aniline with
(R)-2-[3-(4-chlorophenyl)ureido]-3-phenylpropionic acid,
(S)-2-[3-(4-chlorophenyl)ureido]-3-phenylpropionic acid,
   analogously to Example 2 gives the following compounds:
(R)-2-[3-(4-chlorophenyl)ureido]-N-[4-(4-BOC-piperazin-1-yl )-phenyl]-3-phenylpropionamide, hydrochloride,
(S)-2-[3-(4-chlorophenyl)ureido]-N-[4-(4-BOC-piperazin-1-yl)-phenyl]-3-phenylpropionamide, hydrochloride,
   and removal of BOC groups therefrom gives
(R)-2-[3-(4-chlorophenyl)ureido]-N-[4-(piperazin-1-yl)-phenyl]-3-phenylpropionamide, hydrochloride, ESI 478,
(S)-2-[3-(4-chlorophenyl)ureido]-N-[4-(piperazin-1-yl)-phenyl]-3-phenylpropionamide, hydrochloride, ESI 478,

### EXAMPLE 23

Reaction of 1-cyclohexylpiperidin-4-ylamine with
(S)-2-[3-(4-chlorophenyl)ureido]-3-phenylpropionic acid,
(R)-2-[3-(4-chlorophenyl)ureido]-3-phenylpropionic acid,
(R)-2-[3-(4-chlorophenyl)ureido]-2-phenylacetic acid,
   analogously to Example 2 gives the following compounds:
(S)-2-[3-(4-chlorophenyl)ureido]-N-[1-cyclohexylpiperidin-4-yl]-3-phenylpropionamide, hydrochloride, ESI 483;
(R)-2-[3-(4-chlorophenyl)ureido]-N-[1-cyclohexylpiperidin-4-yl]-3-phenylpropionamide, hydrochloride, ESI 483;
(R)-2-[3-(4-chlorophenyl)ureido]-N-[1-cyclohexylpiperidin-4-yl]-2-phenylacetamide.

### EXAMPLE 24

Reaction of 4-(morpholin4-yl)aniline with
2-[N-(4-chlorophenyl)carbamoyloxy]acetic acid,
2-[N-(4-chlorophenyl)carbamoyloxy]propionic acid,
2-[N-(4-chlorophenyl)carbamoyloxy]-2-phenylacetic acid,
   analogously to Example 14 gives the following compounds:
2-[N-(4-chlorophenyl)carbamoyloxy]-N-[4-(morpholin-4-yl)-phenyl]acetamide, ESI 390;
2-[N-(4-chlorophenyl)carbamoyloxy]-N-[4-(morpholin4-yl)-phenyl]propionamide, ESI 404;
2-[N-(4-chlorophenyl)carbamoyloxy]-N-[4-(morpholin-4-yl)-phenyl]-2-phenylacetamide, ESI 466.

### EXAMPLE 25

Reaction of “BB” with
2-[N-(4-chlorophenyl)carbamoyloxy]acetic acid,
2-[N-(4-chlorophenyl)carbamoyloxy]propionic acid,
2-[N-(4-chlorophenyl)carbamoyloxy]-2-phenylacetic acid,
   analogously to Example 14 gives the following compounds:
2-[N-(4-chlorophenyl)carbamoyloxy]-N-(2′-methylsulfonylbiphenyl-4-yl)acetamide, ESI 459;
2-[N-(4-chlorophenyl)carbamoyloxy]-N-(2′-methylsulfonylbiphenyl-4-yl)propionamide, ESI 473;
2-[N-(4-chlorophenyl)carbamoyloxy]-N-(2′-methylsulfonylbiphenyl-4-yl)-2-phenylacetamide.

### EXAMPLE 26

The following are obtained analogously to Example 14:
(R)-2-[N-(4-chlorophenyl)carbamoyloxy]-N-(piperidin-4-ylmethyl)-2-phenylacetamide, trifluoroacetate, ESI 402;
(R)-2-[N-(4-chlorophenyl)carbamoyloxy]-N-(1-isopropylpiperidin-4-yl-methyl)-2-phenylacetamide, hydrochloride, ESI 444;
(R)-2-[N-(4-chlorophenyl)carbamoyloxy]-N-(4-dimethylaminobenzyl)-2-phenylacetamide, ESI 438;
(R)-2-[N-(4-chlorophenyl)carbamoyloxy]-N-[4-(morpholin-4-yl)-benzyl]-2-phenylacetamide, ESI 480;
(R)-2-[N-(4-chlorophenyl)carbamoyloxy]-N-(1-cyclohexylpiperidin-4-yl-methyl)-2-phenylacetamide, hydrochloride, ESI 485;
(R)-2-[N-(4-chlorophenyl)carbamoyloxy]-N-[1-(tetrahydropyran-4-yl )-piperidin-4-ylmethyl]-2-phenylacetamide, hydrochloride, ESI 485;
(R)-2-[N-(4-chlorophenyl)carbamoyloxy]-N-(1-cyclopentylpiperidin-4-yl-methyl)-2-phenylacetamide, hydrochloride, ESI 470;
(R)-2-[N-(4-chlorophenyl)carbamoyloxy]-N-[1-(2-methylpropyl)-piperidin-4-ylmethyl]-2-phenylacetamide, hydrochloride, ESI 458;
(R)-2-[N-(4-chlorophenyl)carbamoyloxy]-N-[1-(1-ethylpropyl)-piperidin-4-ylmethyl]-2-phenylacetamide, hydrochloride, ESI 472;
(R)-2-[N-(4-chlorophenyl)carbamoyloxy]-N-[4-(2-oxo-2H-pyridin-1-yl)-benzyl]-2-phenylacetamide, ESI 488;
(R)-2-[N-(4-chlorophenyl)carbamoyloxy]-N-[4-(2-oxoazepan-1-yl)-phenyl]-2-phenylacetamide, ESI 492;
2-[N-(4-cyanophenyl)carbamoyloxy]-N-[1-(pyridin-4-yl)-piperidin-4-yl-methyl]-2-phenylacetamide, ESI 470;
2-[N-(3-cyanophenyl)carbamoyloxy]-N-[1-(pyridin-4-yl)-piperidin-4-yl-methyl]-2-phenylacetamide, ESI 470;
(R)-2-[N-(4-chlorophenyl)carbamoyloxy]-N-[4-(2-oxopiperidin-1-yl)-phenyl]-2-phenylacetamide, ESI 478;
2-[N-(4-chlorophenyl)carbamoyloxy]-N-[1-(pyridin-4-yl)-piperidin-4-yl-methyl]-2-cyclohexylacetamide, ESI 485;
(R)-2-[N-(4-chlorophenyl)carbamoyloxy]-N-[4-(morpholin-4-yl)-phenyl]-2-phenylacetamide, ESI 466;
2-[N-(4-chlorophenyl)carbamoyloxy]-N-[1-(pyridin-4-yl)-piperidin-4-yl-methyl]-3,3,3-trifluoropropionamide, ESI 471;
(R)-2-[N-(4-chlorophenyl)carbamoyloxy]-N-[4-(piperazin-4-yl)-phenyl]-2-phenylacetamide, ESI 465;
(R)-2-[N-(4-chlorophenyl)carbamoyloxy]-N-[3-(2-oxopiperidin-1-yl)-phenyl]-2-phenylacetamide, ESI 478;
(R)-2-[N-(4-chlorophenyl)carbamoyloxy]-N-[4-(2-oxopiperazin-1-yl)-phenyl]-2-phenylacetamide, ESI 479;
2-[N-(4-chlorophenyl)carbamoyloxy]-N-[1-(pyridin-4-yl)piperidin-4-yl-methyl]-2-(2-thienyl)acetamide, ESI 485.

### EXAMPLE 27

The following are obtained analogously to Example 4:
2-[3-(4-chlorophenyl)ureido]-N-[1-(pyridin-4-yl)piperidin-4-ylmethyl]-2-phenylacetamide, ESI 478;
2-[3-(4-chlorophenyl)ureido]-N-[1-(pyridin-4-yl)piperidin-4-ylmethyl]-4,4,4-trifluorobutyramide, ESI 484;
2-(3-phenylureido)-N-[4-(2-oxopiperidin-1-yl)-phenyl]-3-(4-cyano-phenyl)propionamide, ESI 482;
2-(3-phenylureido)-N-[1-(pyridin-4-yl)piperidin-4-ylmethyl]-3-(3-cyano-phenyl)propionamide, ESI 483;
2-[3-(4-chlorophenyl)ureido]-N-[1-(pyridin-4-yl)piperidin-4-ylmethyl]-3-(3-cyanophenyl)propionamide, ESI 517;
2-(3-phenylureido)-N-[4-(2-oxopiperidin-1-yl)-phenyl]-3-(3-amino-carbonylphenyl)propionamide, ESI 500;
2-(3-phenylureido)-N-[1-(pyridin-4-yl)piperidin-4-ylmethyl]-3-(3-amino-carbonylphenyl)propionamide, ESI 501;
2-[3-(4-chlorophenyl)ureido]-N-[1-(pyridin-4-yl)piperidin-4-ylmethyl]-3-(3-aminocarbonylphenyl)propionamide, ESI 535;
(R)-2-[3-(4-chlorophenyl)ureido]-N-[4-(2-oxopiperidin-1-yl)-phenyl]-2-phenylacetamide, ESI 477;
(R)-2-[3-(4-chlorophenyl)ureido]-N-[3-methyl-4-(2-oxopiperidin-1-yl)-phenyl]-2-phenylacetamide, ESI 491;
(R)-2-[3-(4-chlorophenyl)ureido]-N-[4-(2-oxopiperazin-1-yl)-phenyl]-2-phenylacetamide, ESI 478;
2-[3-(4-chlorophenyl)ureido]-N-[1-(pyridin-4-yl)piperidin-4-ylmethyl]-2-(2-thienyl)acetamide, ESI 484;
2-[3-(4-chlorophenyl)ureido]-N-[4-(2-oxopiperazin-1-yl)-phenyl]-2-(2-thienyl)acetamide, ESI 484;
2-[3-(4-chlorophenyl)ureido]-N-[4-(2-oxo-2H-pyrazin-1-yl)-phenyl]-2-(2-thienyl)acetamide, ESI 480;
(R)-2-[3-(4-chlorophenyl)ureido]-N-[1-isopropylpiperidin-4-ylmethyl]-2-(2-thienyl)acetamide, ESI 449.

The following examples relate to medicaments:

### EXAMPLE A

### Injection Vials

A solution of 100 g of an active ingredient of the formula I and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions. Each injection vial contains 5 mg of active ingredient.

### EXAMPLE B

### Suppositories

A mixture of 20 g of an active ingredient of the formula I with 100 g of soya lecithin and 1400 g of cocoa butter is melted, poured into moulds and allowed to cool. Each suppository contains 20 mg of active ingredient.

### EXAMPLE C

### Solution

A solution is prepared from 1 g of an active ingredient of the formula I, 9.38 g of NaH₂PO₄.2H₂O, 28.48 g of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D

### Ointment

500 mg of an active ingredient of the formula I are mixed with 99.5 g of Vaseline under aseptic conditions.

### EXAMPLE E

### Tablets

A mixture of 1 kg of active ingredient of the formula 1, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### EXAMPLE F

### Coated Tablets

Tablets are pressed analogously to Example E and subsequently coated in a conventional manner with a coating of sucrose, potato starch, talc, tragacanth and dye.

### EXAMPLE G

### Capsules

2 kg of active ingredient of the formula I are introduced into hard gelatine capsules in a conventional manner in such a way that each capsule contains 20 mg of the active ingredient.

### EXAMPLE H

### Ampoules

A solution of 1 kg of active ingredient of the formula I in 60 l of bidistilled water is sterile filtered, transferred into ampoules, lyophilised under sterile conditions and sealed under sterile conditions. Each ampoule contains 10 mg of active ingredient.

1. A compound of the formula I 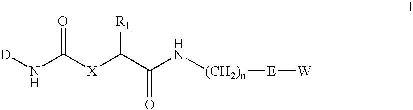
wherein
D is phenyl or pyridyl, each of which is unsubstituted or monosubstituted or polysubstituted by Hal, A, OR², N(R²)₂, NO₂, CN, COOR² or CON(R²)₂,
R¹ is H, Ar, Het, cycloalkyl or A, which may be substituted by OR², SR², N(R²)₂, Ar, Het, cycloalkyl, CN, COOR² or CON(R²)₂,
R² is H or A,
E is phenylene, which may be monosubstituted or polysubstituted by Hal, A, OR², N(R²)₂, NO₂, CN, COOR² or CON(R²)₂,
or is piperidine-1,4-diyl,
W is Ar, Het or N(R²)₂
and, if E=piperidine-1,4-diyl, is alternatively R² or cycloalkyl,
X is NH or O,
A is unbranched or branched alkyl having 1-10 carbon atoms, in which one or two CH₂ groups may be replaced by O or S atoms and/or by —CH═CH— groups and/or in addition 1-7H atoms may be replaced by F,
Ar is phenyl which is unsubstituted or monosubstituted, disubstituted or trisubstituted by Hal, A, OR², N(R²)₂, NO₂, CN, COOR², CON(R²)₂, NR²COA, NR²SO₂A, COR², SO₂NR², SO₃H or S(O)ₘA,
Het is a monocyclic or bicyclic, saturated, unsaturated or aromatic heterocyclic radical having from 1 to 4 N, O and/or S atoms, which may be unsubstituted or monosubstituted, disubstituted or trisubstituted by Hal, A, OR², N(R²)₂, NO₂, CN, COOR², CON(R²)₂, NR²COA, NR²SO₂A, COR², SO₂NR², SO₃H, S(O)ₘA and/or carbonyl oxygen,
Hal is F, Cl, Br or 1,
n is 0 or 1, and
m is 0, 1 or 2,
or a pharmaceutically acceptable salt, solvate or stereoisomer, including a mixture thereof in all ratios.

2. A compound according to claim 1 wherein D is phenyl which is unsubstituted or monosubstituted or disubstituted by Hal, A, OR² or COOR², or pyridyl which is unsubstituted or monosubstituted by Hal.

3. A compound according to claim 1 wherein
Het is a monocyclic or bicyclic, saturated, unsaturated or aromatic heterocyclic radical having from 1 to 2 N, O and/or S atoms, which may be unsubstituted or monosubstituted by carbonyl oxygen.

4. A compound according to claim 1 wherein
Ar is phenyl which is unsubstituted or monosubstituted, disubstituted or trisubstituted by Hal, A, OR², SO₂A, SO₂NH₂, COOR² or CN.

5. A compound according to claim 1 wherein
D is phenyl which is unsubstituted or monosubstituted or disubstituted by Hal, A, hydroxyl, methoxy, ethoxy, hydroxycarbonyl, methoxycarbonyl or ethoxycarbonyl, or pyridyl which is unsubstituted or monosubstituted by Hal.

6. A compound according to claim 1 wherein
R¹ is H, phenyl or alkyl having 1-6 carbon atoms, which may be substituted by thiophene, imidazole, indole, SR², cycloalkyl or phenyl.

7. A compound according to claim 1 wherein
E is 1,4-phenylene or 1,4-piperidinyl.

8. A compound according to claim 1 wherein
Ar is phenyl which is unsubstituted or monosubstituted, disubstituted or trisubstituted by Hal, A, OR², SO₂A, SO₂NH₂, COOR² or CN,
Het is a monocyclic or bicyclic, saturated, unsaturated or aromatic heterocyclic radical having from 1 to 2 N, O and/or S atoms, which may be unsubstituted or monosubstituted by carbonyl oxygen, and
W is Ar, Het or N(R²)₂ and, if E=piperidine-1,4-diyl, is alternatively R².

9. A compound according to claim 1 wherein
Ar is phenyl which is unsubstituted or monosubstituted by Hal, A, OA, SO₂A, COOR², SO₂NH₂ or CN,
Het is thienyl, imidazolyl, pyridyl, indolyl, piperidinyl, piperazinyl, pyrazinyl, 2-oxo-2H-pyrazin-1-yl, 2-oxo-piperazinyl, morpholinyl, tetrahydropyran-4-yl, 3-oxo-morpholin-4-yl, 2-oxopyrrolidin-1-yl or 2-oxopiperidin-1-yl, and
W is Ar, Het or N(R²)₂
and, if E=piperidine-1,4-diyl, is alternatively R².

10. A compound according to claim 1 wherein
D is phenyl which is unsubstituted or monosubstituted or disubstituted by Hal, A, OR² or COOR², or pyridyl which is unsubstituted or monosubstituted by Hal,
R¹ is H, phenyl or alkyl having 1-6 carbon atoms, which may be substituted by thiophene, imidazole, indole, SR², cycloalkyl or phenyl,
E is 1,4-phenylene or 1,4-piperidinyl,
W is Ar, Het or N(R²)₂,
and, if E=piperidine-1,4-diyl, is alternatively R²,
A is alkyl having 1, 2, 3, 4, 5 or 6 carbon atoms or CF₃,
Ar is phenyl which is unsubstituted or monosubstituted or disubstituted by Hal, A, OA, SO₂A, COOR², SO₂NH₂ or CN,
Het is thienyl, imidazolyl, pyridyl, indolyl, piperidinyl, piperazinyl, 2-oxopiperazinyl, pyrazinyl, 2-oxo-2H-pyrazin-1-yl, morpholinyl, tetrahydropyran-4-yl, 3-oxomorpholin-4-yl, 2-oxopyrrolidin-1-yl or 2-oxopiperidin-1-yl,
Hal is F, Cl or Br, and
m is 1 or 2.

11. A compound according to claim 1 wherein
D is phenyl unsubstituted or monosubstituted by Hal, or pyridyl which is unsubstituted or monosubstituted by Hal,
R¹ is H, phenyl or alkyl having 1-6 carbon atoms, which may be substituted by thiophene, imidazole, indole, SR², cycloalkyl or phenyl,
E is 1,4-phenylene,
W is 2-methylsulfonylphenyl,
A is alkyl having 1, 2, 3, 4, 5 or 6 carbon atoms or CF₃, and
n is 0.

12. A compound according to claim 1 wherein
R¹ is H, phenyl or alkyl having 1-6 carbon atoms, which may be substituted by thiophene, imidazole, indole, SR², cycloalkyl or phenyl,
or phenyl or pyridyl, each of which is monosubstituted by Hal or OH.

13. A compound according to claim 1 wherein
D is phenyl which is unsubstituted or monosubstituted by Hal, or pyridyl which is unsubstituted or monosubstituted by Hal,
R¹ is H, phenyl or alkyl having 1-6 carbon atoms, which may be substituted by thiophene, imidazole, indole, SR², cycloalkyl or phenyl,
E is 1,4-piperidinyl,
W is Het, R² or cycloalkyl,
Het is thienyl, imidazolyl, pyridyl, indolyl, piperidinyl, piperazinyl, 2-oxopiperazinyl, pyrazinyl, 2-oxo-2H-pyrazin-1-yl, morpholinyl, tetrahydropyran-4-yl, 3-oxomorpholin-4-yl or 2-oxopiperidin-1-yl,
A is alkyl having 1, 2, 3, 4, 5 or 6 carbon atoms or CF₃, and
n is 0 or 1.

14. A compound which is
(R)-2-[3-(4-chlorophenyl)ureido]-N-(2′-methylsulfonylbiphenyl-4-yl)-3-phenylpropionamide,
(R)-2-[3-(4-chlorophenyl)ureido]-N-(2′-methylsulfonylbiphenyl-4-yl)-propionamide,
(S)-2-(3-pyridin-2-ylureido)-N-(2′-methylsulfonylbiphenyl-4-yl)pentanoamide,
(R)-2-(3-phenylureido)-N-(2′-methylsulfonylbiphenyl-4-yl)pentanoamide, 2-(3-phenylureido)-N-(2′-methylsulfonylbiphenyl-4-yl)-3-(thiophen-2-yl)-propionamide,
2-(3-phenylureido)-N-(2′-methylsulfonylbiphenyl-4-yl)-3-(3H-imidazol-4-yl)propionamide,
(R)-2-(3-phenylureido)-N-(2′-methylsulfonylbiphenyl-4-yl)hexanoamide,
2-(3-phenylureido)-N-(2′-methylsulfonylbiphenyl-4-yl)-4-methylsulfanylbutyramide,
2-(3-phenylureido)-N-(2′-methylsulfonylbiphenyl-4-yl)-2-phenylacetamide,
(S)-2-[3-(4-chlorophenyl)ureido]-N-(2′-methylsulfonylbiphenyl-4-yl)-3-phenylpropionamide,
(R)-2-[3-(4-methylphenyl)ureido]-N-(2′-methylsulfonylbiphenyl-4-yl)-3-phenylpropionamide,
(R)-2-(3-pyridin-4-ylureido)-N-(2′-methylsulfonylbiphenyl-4-yl)pentanoamide,
(S)-2-(3-pyridin-4-ylureido)-N-(2′-methylsulfonylbiphenyl-4-yl)pentanoamide,
(R)-2-(3-pyridin-2-ylureido)-N-(2′-methylsulfonylbiphenyl-4-yl)pentanoamide,
(S)-2-(3-phenylureido)-N-(2′-methylsulfonylbiphenyl-4-yl)pentanoamide,
(R)-2-(3-pyridin-3-ylureido)-N-(2′-methylsulfonylbiphenyl-4-yl)pentanoamide,
(S)-2-(3-phenylureido)-N-(2′-methylsulfonylbiphenyl-4-yl)-3-(pyridin-3-yl)-propionamide,
(S)-2-(3-phenylureido)-N-(2′-methylsulfonylbiphenyl-4-yl)-3-(indol-3-yl)-propionamide,
2-(3-phenylureido)-N-(2′-methylsulfonylbiphenyl-4-yl)propionamide,
2-(3-phenylureido)-N-(2′-methylsulfonylbiphenyl-4-yl)acetamide,
(S)-2-[3-(3-chlorophenyl)ureido]-N-(2′-methylsulfonylbiphenyl-4-yl)-3-phenylpropionamide,
(S)-2-[3-(4-trifluoromethylphenyl)ureido]-N-(2′-methylsulfonylbiphenyl-4-yl)-3-phenylpropionamide,
(S)-2-[3-(2-chlorophenyl)ureido]-N-(2′-methylsulfonylbiphenyl-4-yl)-3-phenylpropionamide,
(S)-2-[3-(4-ethoxyphenyl)ureido]-N-(2′-methylsulfonylbiphenyl-4-yl)-3-phenylpropionamide,
(S)-2-[3-(4-methylphenyl)ureido]-N-(2′-methylsulfonylbiphenyl-4-yl)-3-phenylpropionamide,
(S)-2-[3-(2-methoxyphenyl)ureido]-N-(2′-methylsulfonylbiphenyl-4-yl)-3-phenylpropionamide,
(S)-2-[3-(4-ethoxycarbonylphenyl)ureido]-N-(2′-methylsulfonylbiphenyl-4-yl)-3-phenylpropionamide,
(R)-2-[3-(3-chlorophenyl)ureido]-N-(2′-methylsulfonylbiphenyl-4-yl)-3-phenylpropionamide,
(R)-2-[3-(4-trifluoromethylphenyl)ureido]-N-(2′-methylsulfonylbiphenyl-4-yl)-3-phenylpropionamide,
(R)-2-[3-(2-chlorophenyl)ureido]-N-(2′-methylsulfonylbiphenyl-4-yl)-3-phenylpropionamide,
(R)-2-[3-(4-ethoxyphenyl)ureido]-N-(2′-methylsulfonylbiphenyl-4-yl)-3-phenylpropionamide,
(R)-2-[3-(2-methoxyphenyl)ureido]-N-(2′-methylsulfonylbiphenyl-4-yl)-3-phenylpropionamide,
(R)-2-[3-(4-ethoxycarbonylphenyl)ureido]-N-(2′-methylsulfonylbiphenyl-4-yl)-3-phenylpropionamide,
2-(3-phenylureido)-N-(2′-methylsulfonylbiphenyl-4-yl)-5-BOC-amino-valeramide,
(S)-2-(3-phenylureido)-N-(2′-methylsulfonylbiphenyl-4-yl)-3-phenylpropionamide,
(R)-2-(3-phenylureido)-N-(2′-methylsulfonylbiphenyl-4-yl)-3-phenylpropionamide,
(R)-2-[3-(4-chlorophenyl)ureido]-N-(2′-methylsulfonylbiphenyl-4-yl)-3-cyclopropylpropionamide,
2-[3-(4-chlorophenyl)ureido]-N-(2′-methylsulfonylbiphenyl-4-yl)-4-methylsulfanylbutyramide,
(R)-2-[3-(4-chlorophenyl)ureido]-N-(2′-methylsulfonylbiphenyl-4-yl)-propionamide,
2-[3-(4-chlorophenyl)ureido]-N-(2′-methylsulfonylbiphenyl-4-yl)acetamide,
2-[3-(5-chloro-pyridin-2-yl)ureido]-N-(2′-methylsulfonylbiphenyl-4-yl)-3-phenylpropionamide,
(R)-2-[3-(4-bromophenyl)ureido]-N-(2′-methylsulfonylbiphenyl-4-yl)-3-phenylpropionamide,
(R)-2-[3-(3-fluoro-4-methoxyphenyl)ureido]-N-(2′-methylsulfonylbiphenyl-4-yl)-3-phenylpropionamide,
2-[3-(4-chlorophenyl)ureido]-N-(2′-methylsulfonylbiphenyl-4-yl)hexanoamide,
(R)-2-[3-(4-chlorophenyl)ureido]-N-(2′-methylsulfonylbiphenyl-4-yl)-2-phenylacetamide,
(S)-2-[3-(4-chlorophenyl)ureido]-N-(2′-methylsulfonylbiphenyl-4-yl)-4-methylpentanoamide,
(R)-2-[3-(4-chlorophenyl)ureido]-N-(2′-methylsulfonylbiphenyl-4-yl)-4-methylpentanoamide,
(S)-2-[3-(4-methoxyphenyl)ureido]-N-(2′-methylsulfonylbiphenyl-4-yl)-3-phenylpropionamide,
(S)-2-[3-(4-bromophenyl)ureido]-N-(2′-methylsulfonylbiphenyl-4-yl )-3-phenylpropionamide,
(S)-2-[3-(4-iodophenyl)ureido]-N-(2′-methylsulfonylbiphenyl-4-yl)-3-phenylpropionamide,
(S)-2-[3-(4-fluorophenyl)ureido]-N-(2′-methylsulfonylbiphenyl-4-yl)-3-phenylpropionamide,
(S)-2-[3-(3-fluoro-4-methoxyphenyl)ureido]-N-(2′-methylsulfonylbiphenyl-4-yl )-3-phenylpropionamide,
(R)-2-[3-(4-methoxyphenyl)ureido]-N-(2′-methylsulfonylbiphenyl-4-yl)-3-phenylpropionamide,
(R)-2-[3-(4-bromophenyl)ureido]-N-(2′-methylsulfonylbiphenyl-4-yl)-3-phenylpropionamide,
(R)-2-[3-(4-iodophenyl)ureido]-N-(2′-methylsulfonylbiphenyl-4-yl)-3-phenylpropionamide,
(R)-2-[3-(4-fluorophenyl)ureido]-N-(2′-methylsulfonylbiphenyl-4-yl)-3-phenylpropionamide,
(S)-2-[3-(3-trifluoromethylphenyl)ureido]-N-(2′-methylsulfonylbiphenyl-4-yl)-3-phenylpropionamide,
(R)-2-[3-(3-trifluoromethylphenyl)ureido]-N-(2′-methylsulfonylbiphenyl-4-yl)-3-phenylpropionamide,
2-(3-phenylureido)-N-(2′-methylsulfonylbiphenyl-4-yl)-5-aminovaleramide,
(S)-2-(3-phenylureido)-N-[4-(morpholin-4-yl)-phenyl]-3-phenylpropionamide,
2-(3-phenylureido)-N-[4-(morpholin-4-yl)-phenyl]valeramide,
(R)-2-(3-phenylureido)-N-[4-(morpholin-4-yl)-phenyl]-3-phenylpropionamide,
2-(3-phenylureido)-N-[4-(morpholin-4-yl)-phenyl]-3-(3-cyanophenyl)-propionamide,
2-[3-(4-chlorophenyl)ureido]-N-[4-(morpholin-4-yl)-phenyl]caproamide,
2-[3-(4-chlorophenyl)ureido]-N-[4-(morpholin-4-yl)-phenyl]-4-methyl-sulfanylbutyramide,
(R)-2-[3-(4-chlorophenyl)ureido]-N-[4-(morpholin-4-yl)-phenyl]propionamide,
(S)-2-[3-(4-chlorophenyl)ureido]-N-[4-(morpholin-4-yl)phenyl]-4-methylvaleramide,
(R)-2-[3-(4-chlorophenyl)ureido]-N-[4-(morpholin-4-yl)phenyl]-4-methylvaleramide,
(S)-2-(3-phenylureido)-N-[1-(pyridin-4-yl )-piperidin-4-ylmethyl]-3-phenylpropionamide,
(R)-2-(3-phenylureido)-N-[1-(pyrid in-4-yl)-piperidin-4-ylmethyl]-3-phenylpropionamide,
2-(3-phenylureido)-N-[1-(pyridin-4-yl)-piperidin-4-ylmethyl]valeramide,
(S)-2-[3-(4-chlorophenyl)ureido]-N-[1-(pyridin-4-yl)piperidin-4-ylmethyl]-2-phenylacetamide,
(R)-2-[3-(4-chlorophenyl)ureido]-N-[1-(pyridin-4-yl)piperidin-4-yl-methyl]caproamide,
2-[3-(4-chlorophenyl)ureido]-N-[1-(pyridin-4-yl)piperidin-4-ylmethyl]-4-methylsulfanylbutyramide,
(R)-2-[3-(4-chlorophenyl)ureido]-N-[1-(pyridin-4-yl)piperidin-4-yl-methyl]propionamide,
2-[3-(4-chlorophenyl)ureido]-N-[1-(pyridin-4-yl)piperidin-4-ylmethyl]-3-(thiophen-2-yl)propionamide,
2-[3-(4-chlorophenyl)ureido]-N-[1-(pyridin-4-yl)piperidin-4-ylmethyl]-3-(indol-3-yl)propionamide,
2-[3-(4-chlorophenyl)ureido]-N-[1-(pyridin-4-yl)piperidin-4-ylmethyl]-valeramide,
(S)-2-[3-(4-chlorophenyl)ureido]-N-[1-(pyridin-4-yl)piperid in-4-ylmethyl]-4-methylvaleramide,
(R)-2-[3-(4-chlorophenyl)ureido]-N-[1-(pyridin-4-yl)piperidin-4-ylmethyl]-4-methylvaleramide,
(R)-2-[3-(4-chlorophenyl)ureido]-N-[1-(pyridin-4-yl)piperidin-4-ylmethyl]-2-phenylacetamide,
(R)-2-[3-(4-chlorophenyl)ureido]-N-[1-(pyridin-4-yl)piperidin-4-ylmethyl]-4-methylbutyramide,
(S)-2-[3-(4-chlorophenyl)ureido]-N-[1-(pyridin-4-yl)piperidin-4-ylmethyl]-4-methylbutyramide,
(R)-2-[3-(3-chloropyridin-6-yl)ureido]-N-[1-(pyridin-4-yl)piperidin-4-ylmethyl]-2-phenylacetamide,
2-[3-(4-chlorophenyl)ureido]-N-[1-(pyridin-4-yl)piperidin-4-ylmethyl]-3,3,3-trifluoropropionamide,
2-[3-(4-chlorophenyl)ureido]-N-[1-(pyridin-4-yl)piperidin-4-ylmethyl]-2-(pyridin-2-yl)acetamide,
(R)-2-[3-(4-chlorophenyl)ureido]-N-[1-(pyridin-4-yl)piperidin-4-ylmethyl]-2-(tert-butyl)acetamide,
(S)-2-[3-(4-chlorophenyl)ureido]-N-[1-(pyridin-4-yl)piperidin-4-ylmethyl]-2-(tert-butyl)acetamide,
2-[3-(4-chlorophenyl)ureido]-N-[1-(pyridin-4-yl)piperidin-4-ylmethyl]-2-(2-fluorophenyl)acetamide,
(R)-2-[3-(4-chlorophenyl)ureido]-N-[1-(pyridin-4-yl)piperidin-4-ylmethyl]-2-(4-fluorophenyl)acetamide,
(S)-2-[3-(4-chlorophenyl)ureido]-N-[1-(pyridin-4-yl)piperidin-4-ylmethyl]-2-(4-fluorophenyl)acetamide,
(R)-2-[3-(4-chlorophenyl)ureido]-N-[1-(pyrid in-4-yl )piperidin-4-ylmethyl]-2-(4-hydroxyphenyl)acetamide,
(S)-2-[3-(4-chlorophenyl)ureido]-N-[1-(pyridin-4-yl)piperidin-4-ylmethyl]-2-(4-hydroxyphenyl)acetamide,
2-[3-(4-chlorophenyl)ureido]-N-[1-(pyridin-4-yl)piperidin-4-ylmethyl]-acetamide,
(S)-2-[3-(4-chlorophenyl)ureido]-N-[1-(pyridin-4-yl)piperidin-4-ylmethyl]-3-phenylpropionamide,
(R)-2-[3-(4-chlorophenyl)ureido]-N-[1-(pyridin-4-yl)piperidin-4-ylmethyl]-3-phenylpropionamide,
2-[3-(3-chloropyridin-6-yl)ureido]-N-[1-(pyridin-4-yl)piperidin-4-ylmethyl]-2-(2,1,3-benzothiadiazol-5-yl)acetamide,
(S)-2-(3-phenylureido)-N-(biphenyl-2-ylmethyl)-3-phenylpropionamide,
(R)-2-(3-phenylureido)-N-(biphenyl-2-ylmethyl)-3-phenylpropionamide,
2-(3-phenylureido)-N-(biphenyl-2-ylmethyl)valeramide,
(S)-2-(3-phenylureido)-N-(2′-methylsulfonylbiphenyl-4-ylmethyl)-3-phenylpropionamide,
(R)-2-(3-phenylureido)-N-(2′-methylsulfonylbiphenyl-4-ylmethyl)-3-phenylpropionamide,
2-(3-phenylureido)-N-(2′-methylsulfonylbiphenyl-4-ylmethyl)valeramide,
(R)-2-[3-(4-chlorophenyl)ureido]-N-[1-(pyridin-4-yl)piperidin-4-yl]-3-phenylpropionamide,
(R)-2-[3-(4-chlorophenyl)ureido]-N-[1-(pyridin-4-yl)piperidin-4-yl]-2-phenylacetamide,
2-[3-(4-chlorophenyl)ureido]-N-[1-(pyridin-4-yl)piperidin-4-yl]pentanoamide,
(S)-2-[3-(4-chlorophenyl)ureido]-N-[1-(pyridin-4-yl )piperidin-4-yl]-3-phenylpropionamide,
(R)-2-[3-(4-chlorophenyl)ureido]-N-(2′-tert-butyl-aminosulfonylbiphenyl-4-yl)-3-phenylpropionamide,
(R)-2-[3-(4-chlorophenyl)ureido]-N-(2′-aminosulfonylbiphenyl-4-yl )-3-phenylpropionamide,
(R)-2-[3-(4-chlorophenyl)ureido]-N-[1-(tetrahydropyran-4-yl)-piperidin-4-yl]-3-phenylpropionamide,
(R)-2-[3-(4-chlorophenyl)ureido]-N-[1-isopropylpiperidin-4-yl]-3-phenylpropionamide,
(S)-2-[3-(4-chlorophenyl)ureido]-N-[1-isopropylpiperidin-4-yl]-3-phenylpropionamide,
2-[3-(4-chlorophenyl)ureido]-N-[1-isopropylpiperidin-4-yl]valeramide,
(S)-2-[3-(4-chlorophenyl)ureido]-N-[1-isopropylpiperidin-4-yl]-2-phenylacetamide,
(R)-2-[3-(4-chlorophenyl)ureido]-N-[1-(tetrahydropyran-4-yl)piperidin-4-ylmethyl]-3-phenylpropionamide,
(R)-2-[3-(4-chlorophenyl)ureido]-N-[1-(tetrahydropyran-4-yl)piperidin-4-ylmethyl]-2-phenylacetamide,
(R)-2-[3-(4-chlorophenyl)ureido]-N-[4-(2-oxopiperidin-1-yl)phenyl]-3-phenylpropionamide,
(S)-2-[3-(4-chlorophenyl)ureido]-N-[4-(2-oxopiperidin-1-yl)phenyl]-3-phenylpropionamide,
(R)-2-[3-(4-chlorophenyl)ureido]-N-[4-(3-oxo-morpholin-4-yl)phenyl]-3-phenylpropionamide,
2-[N-(4-chlorophenyl)-carbamoyloxy]-N-[1-(pyridin-4-yl)piperidin-4-yl-methyl]-2-phenylacetamide,
(S)-2-[N-(4-chlorophenyl)-carbamoyloxy]-N-[1-(pyridin-4-yl)piperidin-4-ylmethyl]-2-phenylacetamide,
(R)-2-[N-(4-chlorophenyl)-carbamoyloxy]-N-[1-(pyridin-4-yl)piperidin-4-ylmethyl]-2-phenylacetamide,
2-[N-(4-chlorophenyl)-carbamoyloxy]-N-[1-(pyridin-4-yl)piperidin-4-yl-methyl]acetamide,
2-[N-(4-chlorophenyl)-carbamoyloxy]-N-[1-(pyridin-4-yl)piperidin-4-yl-methyl]propionamide,
2-[N-(4-chlorophenyl)-carbamoyloxy]-2-(2-fluorophenyl)-N-[1-(pyridin-4-yl)piperidin-4-ylmethyl]acetamide,
2-[N-(4-chlorophenyl)-carbamoyloxy]-2-(4-chlorophenyl)-N-[1-(pyridin-4-yl)piperidin-4-ylmethyl]acetamide,
2-[N-(4-chlorophenyl)-carbamoyloxy]-2-(2-chlorophenyl)-N-[1-(pyridin-4-yl)piperidin-4-ylmethyl]acetamide,
(R)-2-[N-(4-chlorophenyl)-carbamoyloxy]-2-(3-chlorophenyl)-N-[1-(pyridin-4-yl)piperidin-4-ylmethyl]acetamide,
(S)-2-[3-(4-chlorophenyl)ureido]-N-[1-cyclopentylpiperidin-4-yl]-3-phenylpropionamide,
(R)-2-[3-(4-chlorophenyl)ureido]-N-[1-cyclopentylpiperidin-4-yl]-3-phenylpropionamide,
(R)-2-[3-(4-chlorophenyl)ureido]-N-[1-cyclopentylpiperidin-4-yl]-2-phenylacetamide,
(R)-2-[3-(4-chlorophenyl)ureido]-N-[4-(2-oxopyrrolidin-1-yl)phenyl]-3-phenylpropionamide,
(S)-2-[3-(4-chlorophenyl)ureido]-N-[4-(2-oxopyrrolidin-1-yl)phenyl]-3-phenylpropionamide,
(R)-2-[3-(4-chlorophenyl)ureido]-N-[4-(piperidin-1-yl)phenyl]-3-phenylpropionamide,
(S)-2-[3-(4-chlorophenyl)ureido]-N-[4-(piperidin-1-yl)phenyl]-3-phenylpropionamide,
(R)-2-[3-(4-chlorophenyl)ureido]-N-[4-(piperidin-1-yl)phenyl]-2-phenylacetamide,
(R)-2-[3-(4-chlorophenyl)ureido]-N-[4-diethylaminophenyl]-3-phenylpropionamide,
(S)-2-[3-(4-chlorophenyl)ureido]-N-[4-diethylaminophenyl]-3-phenylpropionamide,
(R)-2-[3-(4-chlorophenyl)ureido]-N-[4-diethylaminophenyl]-2-phenylacetamide,
(R)-2-[3-(4-chlorophenyl)ureido]-N-[4-dimethylaminophenyl]-3-phenylpropionamide,
(S)-2-[3-(4-chlorophenyl)ureido]-N-[4-dimethylaminophenyl]-3-phenylpropionamide,
(R)-2-[3-(4-chlorophenyl)ureido]-N-[1-(tetrahydropyran-4-yl)piperidin-4-yl]-3-phenylpropionamide,
(S)-2-[3-(4-chlorophenyl)ureido]-N-(1-BOC-piperidin-4-ylmethyl)-3-phenylpropionamide,
(R)-2-[3-(4-chlorophenyl)ureido]-N-(1-BOC-piperidin-4-ylmethyl)-3-phenylpropionamide,
(S)-2-[3-(4-chlorophenyl)ureido]-N-(1-BOC-piperidin-4-ylmethyl)-4-methylpentanoamide,
(R)-2-[3-(4-chlorophenyl)ureido]-N-(1-BOC-piperidin-4-ylmethyl)-4-methylpentanoamide,
(R)-2-[3-(4-chlorophenyl)ureido]-N-(1-BOC-piperidin-4-ylmethyl)-2-phenylacetamide,
(S)-2-[3-(4-chlorophenyl)ureido]-N-(piperidin-4-ylmethyl)-3-phenylpropionamide,
(R)-2-[3-(4-chlorophenyl)ureido]-N-(piperidin-4-ylmethyl)-3-phenylpropionamide,
(S)-2-[3-(4-chlorophenyl)ureido]-N-(piperidin-4-ylmethyl)-4-methylpentanoamide,
(R)-2-[3-(4-chlorophenyl)ureido]-N-(piperidin-4-ylmethyl)-4-methylpentanoamide,
(R)-2-[3-(4-chlorophenyl)ureido]-N-(piperidin-4-ylmethyl)-2-phenylacetamide,
(S)-2-[3-(4-chlorophenyl)ureido]-N-[1-isopropylpiperidin-4-yl]-3-phenylpropionamide,
(R)-2-[3-(4-chlorophenyl)ureido]-N-[1-isopropylpiperidin-4-yl]-3-phenylpropionamide,
(S)-2-[3-(4-chlorophenyl)ureido]-N-[1-isopropylpiperidin-4-yl]-4-methylpentanoamide,
(R)-2-[3-(4-chlorophenyl)ureido]-N-[1-isopropylpiperidin-4-yl]-4-methylpentanoamide,
(R)-2-[3-(4-chlorophenyl)ureido]-N-[1-isopropylpiperidin-4-yl]-2-phenylacetamide,
(R)-2-[3-(4-chlorophenyl)ureido]-N-[1-isopropylpiperidin-4-yl]-2-phenylacetamide,
(R)-2-[3-(4-chlorophenyl)ureido]-N-[4-(4-BOC-piperazin-1-yl)phenyl]-3-phenylpropionamide,
(S)-2-[3-(4-chlorophenyl)ureido]-N-[4-(4-BOC-piperazin-1-yl)phenyl]-3-phenylpropionamide,
(R)-2-[3-(4-chlorophenyl)ureido]-N-[4-(piperazin-1-yl)phenyl]-3-phenylpropionamide,
(S)-2-[3-(4-chlorophenyl)ureido]-N-[4-(piperazin-1-yl)phenyl]-3-phenylpropionamide,
(S)-2-[3-(4-chlorophenyl)ureido]-N-[1-cyclohexylpiperidin-4-yl]-3-phenylpropionamide,
(R)-2-[3-(4-chlorophenyl)ureido]-N-[1-cyclohexylpiperidin-4-yl]-3-phenylpropionamide,
(R)-2-[3-(4-chlorophenyl)ureido]-N-[1-cyclohexylpiperidin-4-yl]-2-phenylacetamide,
2-[N-(4-chlorophenyl)carbamoyloxy]-N-[4-(morpholin-4-yl)-phenyl]-acetamide,
2-[N-(4-chlorophenyl)carbamoyloxy]-N-[4-(morpholin-4-yl)-phenyl]-propionamide,
2-[N-(4-chlorophenyl)carbamoyloxy]-N-[4-(morpholin-4-yl)-phenyl]-2-phenylacetamide,
2-[N-(4-chlorophenyl)carbamoyloxy]-N-(2′-methylsulfonylbiphenyl-4-yl)-acetamide,
2-[N-(4-chlorophenyl)carbamoyloxy]-N-(2′-methylsulfonylbiphenyl-4-yl)-propionamide,
2-[N-(4-chlorophenyl)carbamoyloxy]-N-(2′-methylsulfonylbiphenyl-4-yl)-2-phenylacetamide,
(R)-2-[N-(4-chlorophenyl)carbamoyloxy]-N-(piperidin-4-yl-methyl)-2-phenylacetamide,
(R)-2-[N-(4-chlorophenyl)carbamoyloxy]-N-(1-isopropylpiperidin-4-yl-methyl)-2-phenylacetamide,
(R)-2-[N-(4-chlorophenyl)carbamoyloxy]-N-(4-dimethylamino-benzyl)-2-phenylacetamide,
(R)-2-[N-(4-chlorophenyl)carbamoyloxy]-N-[4-(morpholin-4-yl)-benzyl]-2-phenylacetamide,
(R)-2-[N-(4-chlorophenyl)carbamoyloxy]-N-(1-cyclohexylpiperidin-4-yl-methyl)-2-phenylacetamide,
(R)-2-[N-(4-chlorophenyl)carbamoyloxy]-N-[1-(tetrahydropyran-4-yl)-piperidin-4-yl-methyl]-2-phenylacetamide,
(R)-2-[N-(4-chlorophenyl)carbamoyloxy]-N-(1-cyclopentylpiperidin-4-yl-methyl)-2-phenylacetamide,
(R)-2-[N-(4-chlorophenyl)carbamoyloxy]-N-[1-(2-methyl-propyl)-piperidin-4-yl-methyl]-2-phenylacetamide,
(R)-2-[N-(4-chlorophenyl)carbamoyloxy]-N-[1-(1-ethyl-propyl)piperidin-4-yl-methyl]-2-phenylacetamide,
(R)-2-[N-(4-chlorophenyl)carbamoyloxy]-N-[4-(2-oxo-2H-pyridin-1-yl)-benzyl]-2-phenylacetamide,
(R)-2-[N-(4-chlorophenyl)carbamoyloxy]-N-[4-(2-oxo-azepan-1-yl)-phenyl]-2-phenylacetamide,
2-[N-(4-cyanophenyl)carbamoyloxy]-N-[1-(pyridin-4-yl)piperidin-4-yl-methyl]-2-phenylacetamide,
2-[N-(3-cyanophenyl)carbamoyloxy]-N-[1-(pyridin-4-yl)piperidin-4-yl-methyl]-2-phenylacetamide,
(R)-2-[N-(4-chlorophenyl)carbamoyloxy]-N-[4-(2-oxopiperidin-1-yl)-phenyl]-2-phenylacetamide,
2-[N-(4-chlorophenyl)carbamoyloxy]-N-[1-(pyridin-4-yl)piperidin-4-yl-methyl]-2-cyclohexylacetamide,
(R)-2-[N-(4-chlorophenyl)carbamoyloxy]-N-[4-(morpholin-4-yl)-phenyl]-2-phenylacetamide,
2-[N-(4-chlorophenyl)carbamoyloxy]-N-[1-(pyridin-4-yl)piperidin-4-yl-methyl]-3,3,3-trifluoropropionamide,
(R)-2-[N-(4-chlorophenyl)carbamoyloxy]-N-[4-(piperazin-4-yl)-phenyl]-2-phenylacetamide,
(R)-2-[N-(4-chlorophenyl)carbamoyloxy]-N-[3-(2-oxopiperidin-1-yl)-phenyl]-2-phenylacetamide,
(R)-2-[N-(4-chlorophenyl)carbamoyloxy]-N-[4-(2-oxopiperazin-1-yl)-phenyl]-2-phenylacetamide,
2-[N-(4-chlorophenyl)carbamoyloxy]-N-[1-(pyridin-4-yl)piperidin-4-yl-methyl]-2-(2-thienyl)acetamide,
2-[3-(4-chlorophenyl)ureido]-N-[1-(pyridin-4-yl )piperidin-4-ylmethyl]-2-phenylacetamide,
2-[3-(4-chlorophenyl)ureido]-N-[1-(pyridin-4-yl)piperidin-4-ylmethyl]-4,4,4-trifluorobutyramide,
2-(3-phenylureido)-N-[4-(2-oxopiperidin-1-yl)phenyl]-3-(4-cyanophenyl)-propionamide,
2-(3-phenylureido)-N-[1-(pyridin-4-yl)piperidin-4-ylmethyl]-3-(3-cyano-phenyl)propionamide,
2-[3-(4-chlorophenyl)ureido]-N-[1-(pyridin-4-yl)piperidin-4-ylmethyl]-3-(3-cyanophenyl)propionamide,
2-(3-phenylureido)-N-[4-(2-oxopiperidin-1-yl )phenyl]-3-(3-aminocarbonylphenyl)propionamide,
2-(3-phenylureido)-N-[1-(pyridin-4-yl )piperidin-4-ylmethyl]-3-(3-aminocarbonylphenyl)propionamide,
2-[3-(4-chlorophenyl)ureido]-N-[1-(pyridin-4-yl)piperidin-4-ylmethyl]-3-(3-aminocarbonylphenyl)propionamide,
(R)-2-[3-(4-chlorophenyl)ureido]-N-[4-(2-oxopiperidin-1-yl)phenyl]-2-phenylacetamide,
(R)-2-[3-(4-chlorophenyl)ureido]-N-[3-methyl-4-(2-oxopiperidin-1-yl)-phenyl]-2-phenylacetamide,
(R)-2-[3-(4-chlorophenyl)ureido]-N-[4-(2-oxopiperazin-1-yl)phenyl]-2-phenylacetamide,
2-[3-(4-chlorophenyl)ureido]-N-[1-(pyridin-4-yl)piperidin-4-ylmethyl]-2-(2-thienyl)acetamide,
2-[3-(4-chlorophenyl)ureido]-N-[4-(2-oxopiperazin-1-yl)phenyl]-2-(2-thienyl)acetamide,
2-[3-(4-chlorophenyl)ureido]-N-[4-(2-oxo-2H-pyrazin-1-yl)phenyl]-2-(2-thienyl)acetamide,
(R)-2-[3-(4-chlorophenyl)ureido]-N-[1-isopropylpiperid in-4-ylmethyl]-2-(2-thienyl)acetamide,
or a pharmaceutically acceptable salt, solvate or stereoisomer, including a mixture thereof in all ratios.

15. A process for the preparation of a compound of the formula I according to claim 1 or a pharmaceutically acceptable salt, solvate or stereoisomer, comprising reacting:
a) a compound of the formula II 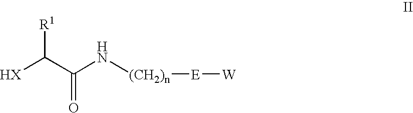
with a compound of the formula III
D-N═C=O III
or
b) reacting a compound of the formula IV
H₂N—(CH₂)ₙ—E—W IV,
with a compound of the formula V 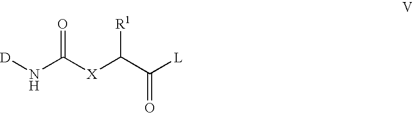
wherein
L is Cl, Br, I or a free or reactively functionally modified OH group, or
d) liberating a compound of the formula I from a functional derivative by treatment with a solvolysing or hydrogenolysing agent,
or
c) converting a base or acid of the formula I into a salt.

16. An inhibitor of a coagulation factor Xa comprising a compound of the formula I according to claim 1

17. An inhibitor of a coagulation factor VIIa comprising a compound of the formula I according to claim 1.

18. A pharmaceutical composition comprising a compound of claim 1 and a pharmaceutically acceptable carrier.

19. A pharmaceutical composition of claim 19 further comprising at least one further pharmaceutically active ingredient.

20. A method of treating thrombosis, myocardial infarction, arteriosclerosis, inflammation, apoplexia, angina pectoris, restenosis after angioplasty, claudicatio intermittens, a tumor, a tumor disease and/or tumor metastases comprising administering an effective amount of a compound according to claim 1 to a patient in need thereof.

21. A set or kit consisting of separate packs of
(a) an effective amount of a compound of the formula I according to claim 1, and
(b) an effective amount of a further pharmaceutically active ingredient.

22. A method for treating thrombosis, myocardial infarction, arteriosclerosis, inflammation, apoplexia, angina pectoris, restenosis after angioplasty, claudicatio intermittens, a tumor, a tumor disease and/or tumor metastases, comprising administering an effective amount of a compound according to claim 1 and at least one further pharmaceutically active ingredient to a patient in need thereof.

23. A pharmaceutical composition according to claim 22, wherein the at least one further pharmaceutically active ingredient is one or more vitamins.

24. A compound of the formula I 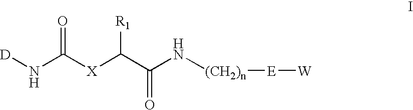
wherein
D is phenyl or pyridyl, each of which is unsubstituted or monosubstituted or polysubstituted by Hal, A, OR², N(R²)₂, NO₂, CN, COOR² or CON(R²)₂,
R¹ is H, Ar, Het, cycloalkyl or A, which may be substituted by OR², SR², N(R²)₂, Ar, Het, cycloalkyl, CN, COOR² or CON(R²)₂,
R² is H or A,
E is phenylene, which may be monosubstituted or polysubstituted by Hal, A, OR², N(R²)₂, NO₂, CN, COOR² or CON(R²)₂, or is piperidine-1,4-diyl,
W is Ar, Het or N(R²)₂ and if E=piperidine-1,4-diyl, is alternatively R² or cycloalkyl,
X is NH or O,
A is unbranched or branched alkyl having 1-10 carbon atoms, in which one or two CH₂ groups may be replaced by O or S atoms and/or by
—CH═CH— groups and/or in addition 1-7H atoms may be replaced by F,
Ar is phenyl which is unsubstituted or monosubstituted, disubstituted or trisubstituted by Hal, A, OR², N(R²)₂, NO₂, CN, COOR², CON(R²)₂, NR²COA, NR²SO₂A, COR², SO₂NR², SO₃H or S(O)ₘA,
Het is a monocyclic or bicyclic, saturated, unsaturated or aromatic heterocyclic radical having from 1 to 4 N, O and/or S atoms, which may be unsubstituted or monosubstituted, disubstituted or trisubstituted by Hal, A, OR², N(R²)₂, NO₂, CN, COOR², CON(R²)₂, NR²COA, NR²SO₂A, COR², SO₂NR², SO₃H, S(O)ₘA and/or carbonyl oxygen,
Hal is F, Cl, Br or I,
n is 0 or 1, and
m is 0, 1 or 2,
or a pharmaceutically acceptable derivative, solvate or stereoisomer including a mixture thereof in all ratios.

25. A compound according to claim 1, wherein:
D is phenyl which is unsubstituted or monosubstituted by Hal, or pyridyl which is unsubstituted or monosubstituted by Hal,
R¹ is H, phenyl or alkyl having 1-6 carbon atoms, which may be substituted by thiophene, imidazole, indole, SR², cycloalkyl or phenyl,
E is 1,4-piperidinyl,
W is Het,
Het is thienyl, imidazolyl, pyridyl, indolyl, piperidinyl, piperazinyl, 2-oxopiperazinyl, pyrazinyl, 2-oxo-2H-pyrazin-1-yl, morpholinyl, tetrahydropyran-4-yl, 3-oxomopholin-4-yl or 2-oxopiperidin-1-yl, and
A is alkyl having 1, 2, 3, 4, 5, or 6 carbon atoms or CF₃.
